(19) United States
(12) Patent Application Publication
(10) Pub. No.: US 2025/0025484 A1
(43) Pub. Date: Jan. 23, 2025

(54) POLYMERSOME NANOPARTICLES EXHAUSTING INTRACELLUAR AMINO ACID FOR CANCER THERAPY AND METHODS THEREOF

(71) Applicant: THE UNIVERSITY OF HONG KONG, HONG KONG (CN)

(72) Inventors: Weiping WANG, Hong Kong (CN); Tianyi WANG, Hong Kong (CN)

(73) Assignee: THE UNIVERSITY OF HONG KONG, HONG KONG (CN)

(21) Appl. No.: 18/778,471

(22) Filed: Jul. 19, 2024

Related U.S. Application Data
(60) Provisional application No. 63/514,949, filed on Jul. 21, 2023.

(51) Int. Cl. A61K 31/69; A61K 9/127; A61K 31/4965; A61P 35/00

(52) U.S. Cl. CPC A61K 31/69; A61K 9/1273; A61K 31/4965; A61P 35/00

(57) ABSTRACT

Disclosed are polymersome nanoparticles that comprise a combination of drugs: (i) a ubiquitin-proteasome system (UPS) inhibitor; and (ii) a macropinocytosis inhibitor. Also disclosed is a method of treatment of cancer using the nanoparticles. Disclosed is a method of treatment of tumor having nutrient-deprived microenvironment, such as deficient in amino acids. The disclosure is directed to a combination of drugs for starvation of tumor cells comprising the use of nanoparticles which inhibits degradation of proteins in the tumor cells and prevents internalization of proteins into the tumor cells. Also provided are compositions and methods for affecting cell proliferation, metabolism or sensitization, including proteasome inhibitor therapy for the treatment of disease caused by deregulation of ubiquitin-proteasome systems, in a cell or subject.

### RELATED APPLICATIONS

The present application claims priority to U.S. Provisional Application Ser. No. 63/514,949 filed Jul. 21, 2023, the content of which is incorporated by reference in its entirety.

### 1. FIELD

The disclosure provides a nanoparticle system to achieve exacerbating amino acid starvation for cancer therapy, which provides a novel strategy for clinical cancer treatment. Also provided is a method of treating cancer using the nanoparticle system. The present application relates to a composition and methods for affecting cell proliferation, metabolism or sensitization, including proteasome inhibitor therapy for the treatment of cancer.

### 2. BACKGROUND

As one of the leading causes of death worldwide, cancer is characterized by uncontrolled cell proliferation and highly progressive nature. Solid tumors often outstrip their blood supply and develop a tumor microenvironment deprived of nutrients, such as amino acids and glucoses (1-3). As the most abundant organic constituents in the body fluid, proteins have the potential to serve as an alternative resource as each protein can provide a large number of amino acids by several orders of magnitude (2). Therefore, cancer cells develop strategies to utilize extracellular proteins. For example, under amino acid starvation, extracellular proteins are internalized through macropinocytosis (2, 4). Mammalian target of rapamycin (mTOR) signaling pathway is inhibited and mTOR complex 1 (mTORC1) is released from the lysosome membrane, leading to vacuolar-type H+ ATPase (V-ATPase) assembly at the lysosomes. As active proton pumps, these V-ATPases decrease lysosomal pH to increase protease activity and promote lysosomal degradation of protein contents (5). Decreased mTOR activity also triggers Unc51-like kinase 1/2 activation, enhancing autophagosome formation and autophagic protein degradation (2, 6). Although the function of lysosomes during this process is demonstrated, it remains largely unknown whether another pathway, ubiquitin-proteasome system (UPS), that accounts for degradation of 80-90% proteins, also contributes to amino acid starvation-induced endocytosed protein degradation (7).

As a primary intracellular protein degradation system, UPS degrades various types of proteins, including short-lived, native, misfolded, or damaged proteins, in a highly selective manner (8). Initially, ubiquitin is activated by binding to the active site cysteine of the ubiquitin-activating enzyme (E1). Subsequently, ubiquitin is transferred to the ubiquitin conjugase (E2) and ultimately conjugated to lysine residue or N-terminal amino group of the substrates by the ubiquitin ligase (E3). Eventually, the protein with polyubiquitinated chains is recognized and degraded by the proteasome (9). UPS controls various basic cellular processes such as cell cycle progression, signal transduction, metabolism, and protein quality control (10, 11). However, it is not thoroughly understood whether UPS is activated by amino acid starvation for extracellular protein degradation to supply amino acid pool for cancer cell survival. Identifying the influence of amino acid starvation on UPS activity may provide a potential target for cancer starvation therapy, as proteasome inhibition may efficiently block nutrient supply in starvation-adapted cancer cells with high UPS activity. Since UPS mediates intracellular protein degradation, proteins in the extracellular space need to be internalized before the proteolytic process. When proteasome inhibition blocks UPS-dependent protein degradation, insufficient intracellular amino acids may trigger compensatory protein internalization, which may lead to resistance to the starvation therapy. Therefore, it is highly desired to both block protein internalization and degradation for cancer starvation therapy.

### 3. SUMMARY

The disclosure provides a method of treating cancer, comprising the step of administering to a subject in need thereof a combination of (i) a ubiquitin-proteasome system (UPS) inhibitor; and (ii) a macropinocytosis inhibitor.

Also provided is a polymersome nanoparticle comprising a ubiquitin-proteasome system (UPS) inhibitor and a macropinocytosis inhibitor.

Also provided is a method of treating a tumor, comprising the step of contacting the tumor with a combination of (i) a ubiquitin-proteasome system (UPS) inhibitor; and (ii) a macropinocytosis inhibitor.

Also provided is a method of reducing protein internalization and degradation in a tumor surrounded by an amino-acid deprived microenvironment, the method comprises the step of contacting the tumor with a combination of (i) a ubiquitin-proteasome system (UPS) inhibitor; and (ii) a macropinocytosis inhibitor.

In certain embodiments, the nanoparticles are modified with tumor-targeting ligands on the surface for enhanced anti-cancer effect.

In one embodiment, provided is a method of treating cancer, comprising the step of administering to a subject in need thereof a combination of: (i) a ubiquitin-proteasome system (UPS) inhibitor; and (ii) a macropinocytosis inhibitor.

In one embodiment, provided is a polymersome nanoparticle comprising a ubiquitin-proteasome system (UPS) inhibitor and a macropinocytosis inhibitor.

In one embodiment, provided is a method of treating a tumor, comprising the step of contacting the tumor with a combination of (i) a ubiquitin-proteasome system (UPS) inhibitor; and (ii) a macropinocytosis inhibitor.

In one embodiment, provided is a method of reducing protein internalization and degradation in a tumor surrounded by an amino-acid deprived microenvironment, the method comprises the step of contacting the tumor with a combination of (i) a ubiquitin-proteasome system (UPS) inhibitor; and (ii) a macropinocytosis inhibitor.

In another aspect, the disclosure provides a kit comprising, (i) a ubiquitin-proteasome system (UPS) inhibitor; and (ii) a macropinocytosis inhibitor; and instruction for use.

### 4. BRIEF DESCRIPTION OF FIGURES

The patent or application file contains at least one drawing executed in color. Copies of this patent or patent application publication with color drawings will be provided by the Office upon request and payment of the necessary fee.

FIGS. 1A-C. Show a schematic illustration depicting the pH-responsive polymersomes loaded with BTZ and EIPA exhausting intracellular amino acids for cancer starvation therapy. Created with BioRender.com. (A) UPS activation by starvation. (B) Polymersome formulation. (C) Polymersome exhausting intracellular amino acids.

FIGS. 2A-J. Amino acid-mediated starvation induces UPS activation for internalized protein degradation. A) Representative CLSM images of FITC-BSA (0.2 mg/mL) internalized by A549 cancer cells treated with DMEM or EBSS medium for 4 h. B) Flow cytometry analysis, and C) quantified result of FITC-BSA (0.2 mg/mL) internalization in A549 cells treated with DMEM or EBSS medium for 4 h. D, E) Western blot analysis of K48-polyubiquitinated proteins in A549 cells treated with DMEM, EBSS, EBSS plus BTZ (10 μM) for 2 h or 24 h. F) Flow cytometry result, and G) quantified analysis of proteasome activity experiment in A549 cells receiving DMEM, EBSS, EBSS plus BTZ (10 μM) for 4 h. H) Representative CLSM images of DQ-BSA (10 μg/mL) internalization and degradation analysis in A549 cells receiving DMEM, EBSS, EBSS plus BTZ (10 μM) for 4 h. I) Flow cytometry result, and J) quantified analysis of DQ-BSA uptake and degradation analysis in A549 cells receiving indicated treatments for 4 h. Data are represented as mean±SD (n=3). **p<0.01, ****p<0.0001.

FIGS. 3A-L. UPS activity in starvation-adapted A549 cells and LUAD tumor tissues. A) Outline of the process for starvation-adapted cell line construction. B) Top 70 DEGs of RNAseq analysis between normal A549 cells and starvation-adapted A549 cells. C) GSEA analysis of all DEGs between normal A549 cells and starvation-adapted A549 cells. D) Flow cytometry analysis of proteasome activity in normal A549 cells and starvation-adapted A549 cells, treated with normal DMEM or EBSS, separately. E) Flow cytometry analysis of FITC-BSA internalization in normal A549 cells and starvation-adapted A549 cells, treated with normal DMEM or EBSS, separately. F) Volcano plot of UPS-associated DEGs between lung tumor tissues and surrounding normal tissues. G) GO enrichment analysis of UPS-associated DEGs between lung tumor tissues and surrounding normal tissues. H) Cell viability analysis of A549 cells treated with DMSO or BTZ (20 μM), in DMEM or EBSS medium for 24 h. I) Western blot analysis of p-S6K (70 kDa), S6K (70 kDa), and mTOR (289 kDa) in A549 cells treated with normal DMEM or EBSS medium for 2 h. J) Western blot analysis of K48-polyubiquitinated proteins in A549 cells treated with DMSO or rapamycin (100 μM) for 2 h. K) Flow cytometry and L) quantified analysis of proteasome activity probe in A549 cells treated with a series of concentrations of rapamycin for 4 h. Data are represented as mean±SD (n=3). **p<0.01, ****p<0.0001, ns: not significant.

FIGS. 4A-L. Synergistic anti-cancer effect of EIPA and BTZ. A) Representative CLSM images of TMR-Dextran internalization in A549 cells treated with DMEM, EBSS, or EBSS plus EIPA (50 μM, pretreated 1 h before) for 1 h. B) Flow cytometry and quantified analysis of TMR-Dextran internalization in A549 cells treated with DMEM, EBSS, or EBSS plus EIPA (50 UM, pretreated 1 h before) for 1 h. C) Representative CLSM images of FITC-BSA internalization in A549 cells treated with DMEM, EBSS, or EBSS plus EIPA (50 μM, pretreated 1 h before) for 1 h. D) Flow cytometry and quantified analysis of FITC-BSA internalization in A549 cells treated with DMEM, EBSS, or EBSS plus EIPA (50 μM, pretreated 1 h before) for 1 h. E) Representative co-localization analysis of FITC-BSA and TMR-Dextran in A549 cells using CLSM. F) Cell viability analysis of A549 cells treated with different concentrations of EIPA and BTZ for 24 h in EBSS medium. G) Flow cytometric apoptosis analysis, and H) quantified result of A549 cells treated with EIPA (50 μM), BTZ (20 μM), or their combination in EBSS medium for 24 h. I) Flow cytometry, and J) quantified analysis of DQ-BSA fluorescence intensity in A549 cells treated with EIPA (50 μM), BTZ (20 μM), or their combination in DMEM or EBSS medium for 24 h. K) The level of GSH in A549 cells treated with EIPA (50 M), BTZ (10 μM), or their combination in DMEM or EBSS (3% BSA) medium for 24 h. L) DCFH-DA flow cytometric analysis in A549 cells treated with EIPA (50 μM), BTZ (10 μM), or their combination in indicated medium for 24 h. Data are represented as mean±SD (n=3). ***p<0.001, ****p<0.0001.

FIGS. 5A-J. Combination of EIPA and BTZ circumvents compensatory protein catabolism. A) Scheme depicting the influence of EIPA on intracellular protein degradation. Created with BioRender.com. B) Flow cytometry and C) quantified analysis of cells pretreated with DQ-BSA for 1 h, then cultured in DMEM, EBSS plus BSA, EBSS plus BSA and EIPA for another 3 h. D) Scheme depicting the effect of BTZ on extracellular protein internalization and lysosome-dependent degradation. Created with BioRender.com. E) Floy cytometry and F) quantified analysis of FITC-BSA internalization in A549 cells receiving DMEM, EBSS, or EBSS plus BTZ for 4 h. G) Representative CLSM images and H, I) quantified analysis of mCherry-GFP-LC3 reporter system and BSA-AF647 internalization in A549 cells treated with DMEM, EBSS, and EBSS plus BTZ for 4 h using FIJI-ImageJ software with ComDet plugin. J) Western blot analysis of SQSTM1 (62 kDa) in A549 cells treated with indicated drugs in DMEM or EBSS plus BSA medium for 4 h. Data are represented as mean±SD (n=3). *p<0.05, **p<0.01, ****p<0.0001.

FIGS. 6A-F. Characterization of polymersomes encapsulating EIPA and BTZ. A) Scheme of the polymersomes encapsulating EIPA and BTZ, which can undergo pH-responsive cleavage. Created with BioRender.com. B) Average sizes of ENPs, BNPs, and EBNPs using DLS. C) TEM image of EBNPs. D) Stability test of ENPs, BNPs, and EBNPs in DMEM with 10% FBS at 37° C. for 5 days. E) Drug release profile of EBNPs for 24 h at pH 7.4 and pH 5.0, separately. F) Cell viability of A549 cells treated with ENPs, BNPs, and EBNPs for 24 h. Concentrations of EIPA was 25μ, 50μ, or 100μ. Concentrations of BTZ was 14μ, 28μ, or 56 μM. Data are represented as mean±SD (n=3). **p<0.01, ***p<0.001.

FIGS. 7A-F. Anti-tumor effect of polymersome nanoparticles in xenograft tumor mouse model. A) Schematic illustration of the schedule for xenograft tumor model implantation and synergistic therapy. B) Images of xenograft tumor tissues excised from tumor-bearing mice at the endpoint of treatments. (n=4) C) Tumor volume profile of tumor-bearing mice receiving different treatments. D) Tumor weight of xenograft tumor tissues excised from tumor-bearing mice at the endpoint of treatments. (n=4) E) Representative H&E staining analysis of tumor tissues excised from tumor-bearing mice at the endpoint of treatments. F) Representative TUNEL assay of tumor tissues excised from tumor-bearing mice at the endpoint of treatments.

FIG. 8 shows 23 UPS genes that may influence prognosis in lung adenocarcinoma patients analyzed by univariate Cox regression.

FIG. 9 shows the expression of PRC1, TRIM6, and TRIML2 may influence the survival of lung adenocarcinoma patients as independent risk factors.

FIG. 10 shows mean synergy score of EIPA and BTZ combination calculated by SynergyFinder website. Three calculating models were applied, including HSA, Bliss, and ZIP models.

FIGS. 11A-B show the synthetic routes of the block copolymers. (A) Formation of THPMA. (B) Formation of PEG₁₁₃-b-P(BzMAₓ-co-THPMA_{y}.

FIG. 12 shows ¹H-NMR spectrum of THPMA.

FIG. 13 shows ¹H-NMR spectrum of PEG₁₁₃-b-P(BzMA₆₈-CO-THPMA₆₃).

FIG. 14 shows pH-responsive cleavage of PEG-b-P(BzMA-co-THPMA) under acidic environment for 24 h.

FIG. 15 shows body weight profiles of the tumor-bearing mice receiving different treatments.

FIG. 16 shows H&E staining profile of major organs from the tumor-bearing mice, including heart, liver, spleen, lung, and kidney, at the end of different treatments. Scale bar: 200 μm.

### 4.1 DEFINITIONS

As used herein, the following terms have the meanings ascribed to them unless specified otherwise.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “administering” refers to an administration that is oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “pharmaceutically acceptable carrier” means a carrier that is useful in preparing a pharmaceutical composition that is generally safe, non-toxic and neither biologically nor otherwise undesirable, and includes a carrier that is acceptable for veterinary use as well as human pharmaceutical use. A “pharmaceutically acceptable carrier” as used in the specification and claims includes both one and more than one such carrier.

The term “pharmaceutically acceptable salts” refers to any acid or base addition salt whose counter-ions are non-toxic to the subject to which they are administered in pharmaceutical doses of the salts.

The terms “effective amount” or “effective dose” refer to the amount of a compound that will elicit the biological or medical response of a tissue, system, animal, or human that is being sought by the researcher, veterinarian, medical doctor or other clinician. The term “therapeutically effective amount” includes that amount of a compound that, when administered alone or together with another compound, is sufficient to prevent development of, or alleviate to some extent, one or more of the symptoms of the condition or disorder being treated. The effective amount will vary depending on the compound, the disorder or conditions and its severity, the route of administration, time of administration, rate of excretion, drug combination, judgment of the treating physician, dosage form, and the age, weight, general health, sex and/or diet of the subject to be treated. In the context of the present method, a pharmaceutically or therapeutically effective amount or dose of includes an amount that is sufficient to treat a disease such as cancer, rheumatoid arthritis, autoimmune disease, transplant rejection, multiple sclerosis, sepsis, inflammatory bowel disease, lupus, proliferative disease, benign tumors, hyperplasias, and cachexia.

The terms “increased effectiveness” as used herein, refer to increasing a therapeutic efficacy of a compound. An increase in the therapeutic efficacy of a compound can be measured by a decrease in the dosage required to achieve a particular therapeutic result, a decrease in the amount of time or number of treatments required to achieve a particular therapeutic result, or an increase in the scope or type of therapeutic results that can be obtained by administration of the compound.

The terms “prevent”, “preventing”, “prevention” and grammatical variations thereof as used herein, refer to a method of partially or completely delaying or precluding the onset or recurrence of a disorder or conditions and/or one or more of its attendant symptoms or barring a subject from acquiring or reacquiring a disorder or condition or reducing a subject's risk of acquiring or reacquiring a disorder or condition or one or more of its attendant symptoms.

The term “proteasome system inhibitor” refers to a compound that inhibits one or more activities or functions of a proteasome. Examples of proteasome inhibitors include, but are not limited to, bortezomib, carfilzomib, disulfiram, epigallocatechin-3-gallate, salinosporamide, MLN 9708, ONX 0912, CEP-1877, NPI-0052, and NLVS, a synthetic active center directed, irreversible proteasome inhibitor (4-hydroxy-5-iodo-3-nitrophenylacetyl-Leu-Leu-leucinal-vinyl sulfone), lactacystin, clasto-beta-lactone, epoxomicin, eponemycin, dihydrocponemycin, MLN 519.

The term “subject” is defined herein to include animals such as mammals, including, but not limited to, primates (e.g., humans), cows, sheep, goats, horses, dogs, cats, rabbits, rats, mice and the like. In preferred embodiments, the subject is a human. The term “subject” also includes animal and human patients.

The terms “therapeutic effect” and “therapeutic efficacy” refer to a biological or medical response of a tissue, system, animal, or human that is being sought by a researcher, veterinarian, medical doctor or other clinician and include the treatment of a disease, disorder, or condition.

The terms “treat”, “treating”, “treatment” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively or remedially. In some instances, the terms “treat”, “treating”, “treatment” and grammatical variations thereof, include partially or completely reducing the size of a tumor or cancerous lesion, reducing the number of tumors or cancerous lesions, and reducing the severity of a tumor or cancerous lesion as compared with prior to treatment of the subject or as compared with the incidence of such symptom in a general or study population.

The terms “synergy”, “synergistic”, “synergism” and the like refer to the production of a greater than expected additive effect of two or more drugs when used in combination. Mathematical analysis of the cytotoxicity data provided above and below in the Examples was performed with CalcuSyn (BioSoft), which confirmed that the combination of a proteasome system inhibitor and a micropinocytosis inhibitor was highly synergistic. CalcuSyn estimates the Dose Reduction Index (DRI), which is a measure of how much the dose of, for example, bortezomib can be reduced at a given effect level relative to the dose of bortezomib alone.

### 5. DETAILED DESCRIPTION

Characterized by uncontrolled cell proliferation and highly progressive nature, solid tumors often outstrip their blood supply and develop a starved microenvironment. The ubiquitin-proteasome system (UPS) plays essential roles in mediating protein degradation. However, it remains unclear if UPS is activated to promote internalized protein degradation under amino acid starvation. Herein, we demonstrate that amino acid starvation can activate protein internalization and facilitate UPS-dependent degradation, sensitizing cancer cells to UPS inhibition. To improve therapeutic efficacy, we combined UPS inhibition with macropinocytosis inhibition to block protein internalization and UPS-dependent degradation simultaneously. To further improve the tumor accumulation of the drugs and reduce the systemic adverse effects, a pH-responsive polymersome nanocarrier was developed to deliver the therapeutic agents to the tumor tissues through enhanced permeability and retention effect. This nanoparticle system provides a novel approach to exacerbate amino acid starvation for cancer therapy, which represents a promising strategy for clinical cancer treatment.

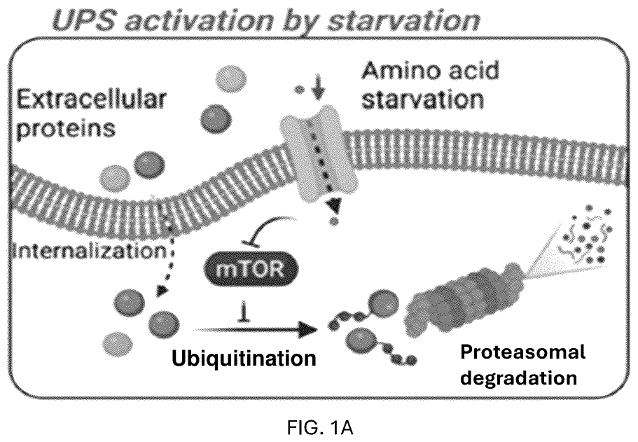

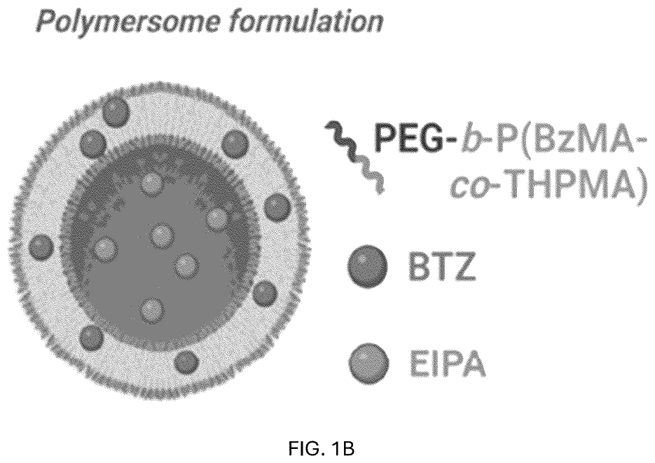

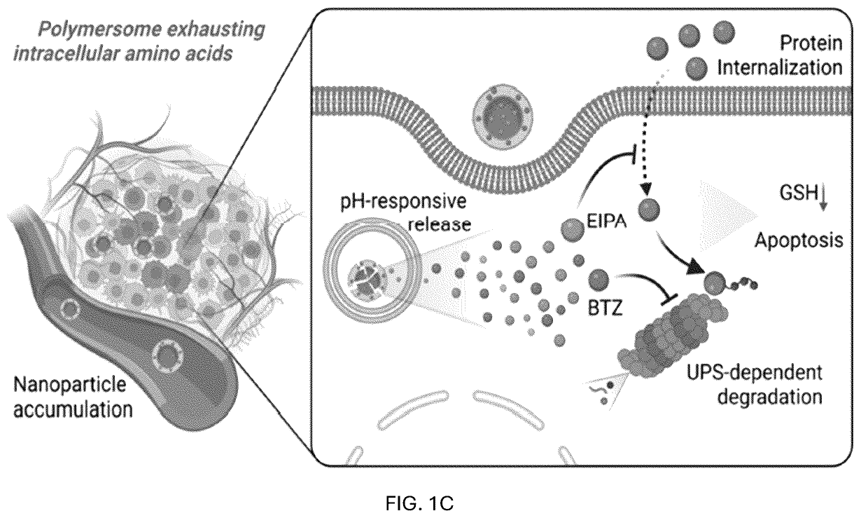

In one example, we evaluated the influence of starvation on UPS in cancer cells. We found amino acid starvation may activate UPS for internalized protein degradation to provide nutrients for cancer cell survival. As a result, starved cancer cells became more sensitive to the UPS inhibition. Therefore, UPS may serve as a novel target for cancer starvation therapy. Our results showed that proteasome inhibition enhanced protein internalization as a feedback, which may provide supplementary nutrients through autophagic degradation. Inhibiting protein internalization by macropinocytosis inhibitor could significantly exacerbate amino acid starvation. Therefore, we explored the potential of the combination therapy with a UPS inhibitor, bortezomib (BTZ), and a macropinocytosis inhibitor, 5-(n-ethyl-n-isopropyl)-amiloride (EIPA) in cancer treatment. To co-deliver both hydrophobic drug, BTZ, and hydrophilic drug, EIPA, a pH-responsive polymersome delivery system was developed to further enhance drug accumulation at tumor tissues and control drug release in cancer cells. In stark contrast with previous nanomedicines consuming glucose or blocking amino acid transporter, for the first time, we developed a coordinative nanocarrier targeting protein catabolismas a strategy for starvation-based cancer therapy (FIG. 1).

### 5.1 UBIQUITIN-PROTEASOME SYSTEM INHIBITOR

The ubiquitin-proteasome system inhibitor can be a proteasome inhibitor or an inhibitor of another component of the ubiquitin-proteasome pathway such an as E3 ubiquitin ligase. Ubiquitin-proteasome system inhibitors include, but are not limited to, bortezomib, carfilzomib, disulfiram, epigallocatechin-3-gallate, salinosporamide, lactacystin, clasto-beta-lactone, epoxomicin, eponemycin, dihydroeponemycin, MLN 519, MLN 9708, ONX 0912, CEP-1877, NPI-0052, NLVS (4-hydroxy-5-iodo-3-nitrophenylacetyl-Leu-Leu-leucinal-vinyl sulfone). In one embodiment, the ubiquitin-proteasome system inhibitor is bortezomib. In another embodiment, the ubiquitin-proteasome system inhibitor is NLVS. Examples of E3 ubiquitin ligase inhibitors include, but are not limited to, MLN 4924, JNJ-26854165, and RG7112.

### 5.2 MACROPINOCYTOSIS INHIBITOR

In some embodiments, micropinocytosis inhibitor is from the group of: 5-(N-ethyl-N-isopropyl) amiloride (EIPA), 3-methylsulphonyl-4-piperidinobenzoyl) guanidine methanesulphonate (HOE-694), amiloride and amiloride derivatives, eganelisib, IPI-549, MBQ-167, wortmannin, LP1-106, XRK3F2, or LY294002. In some embodiments, the macropinocytosis inhibitor is 5-(N-ethyl-N-isopropyl) amiloride, 3-methylsulphonyl-4-piperidinobenzoyl) guanidine methanesulphonate or IPI-549. In some embodiments, the micropinocytosis inhibitor is an inhibitor of activity or expression of one or more proteins selected from NRF2, PIK3CG, CDC42, PAK1, SLC9A1, SNX5, SDC1, NHE1, p110α, p110γ, CDC42 and EGFR or EGF. In some embodiments, the macropinocytosis inhibitor is 5-(N-ethyl-N-isopropyl) amiloride. In some embodiments, the macropinocytosis inhibitor is 3-methylsulphonyl-4-piperidinobenzoyl) guanidine methanesulphonate or IPI-549. In some embodiments, the inhibitor of NRF2 is ML385 or Brusatol. In some embodiments, the inhibitor of EGFR is selected from erlotinib, gefitinib, lapatinib, cetuximab, osimertinib, neratinib, panitumumab, vandetanib, necitumumab, or dacomitinib. In some embodiments, the macropinocytosis inhibitor is an inhibitor of p62 activity or expression. In some embodiments, the macropinocytosis inhibitor comprises LP1-106 or XRK3F2.

In some embodiments, the amount of macropinocytosis inhibitor in the formulation can be from 1.0 mg/mL to 100 mg/mL, about 1.0 mg/mL to about 50 mg/mL protein, about 5.0 mg/mL to about 25 mg/mL. In another embodiment, the amount of macrophage macropinocytosis inhibitor in the formulation can be about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. The composition can be formulated to achieve a concentration of about 9 mg/mL.

In some embodiments, the macrophage macropinocytosis inhibitor composition releases the macrophage macropinocytosis inhibitor at a steady rate for 30, 60, 90, or 120 days after administration.

### 5.3 METHODS OF TREATMENT

In accordance with one of the methods, a therapeutically effective amount of a UPS inhibitor and a micropinocytosis inhibitor are administered to the cell or subject. The UPS inhibitor is administered concurrently with, after, before or between cycles of administration of the micropinocytosis inhibitor. Use of the method results in increased effectiveness the ubiquitin-protease inhibitor, and thereby, improves the efficacy of the ubiquitin-proteasome system inhibitor. Accordingly, the method improves the efficacy of treatments for cancer, rheumatoid arthritis, autoimmune disease, transplant rejection, multiple sclerosis, sepsis, inflammatory bowel disease, lupus, proliferative disease, benign tumors, hyperplasias, and cachexia. The method is particularly useful for treatment of the multiple myeloma.

In accordance with another one of the methods, an effective amount of the compound is administered to a cell or subject to inhibit cell proliferation.

In accordance with yet another one of the methods an effective amount of the compound is administered to a cell or subject to increase, or augment, glucose utilization.

The compound may exist in different isomeric forms. These forms include constitutional, or structural, isomers and stereoisomers. While structural isomers have different bond connections and/or a different order between different atoms or groups, stereoisomers differ in their spatial orientation. Stereoisomers include enantiomers and diastereomers. Diastereomers are further subdivided into cis-trans isomers, conformers, and rotamers.

As mentioned above, a method is provided that includes administering to a cell or subject one or more ubiquitin-proteasome system inhibitors and an effective amount of micropinocytosis inhibitor. Also provided is a method of treating a disease in a subject by administering to the subject a therapeutically effective amount of one or more ubiquitin-proteasome system inhibitor and a micropinocytosis inhibitor.

The ubiquitin-proteasome system inhibitors used in the method include proteasome inhibitors. In some embodiments, the proteasome inhibitor is selected from the group consisting of bortezomib, carfilzomib, MLN 9708, ONX 0912, CEP-1877, NPI-0052, and NLVS. In one embodiment, the proteasome inhibitor is bortezomib, another embodiment. In the proteasome inhibitor is NLVS. Ubiquitin-proteasome inhibitors also include inhibitors of other components of the ubiquitin-proteasome system such as an E3 ubiquitin ligase. Accordingly, the method includes the use of E3 ubiquitin ligase inhibitors such as MLN 4924, JNJ-26854165, and RG7112.

The therapeutic effect using the disclosed method can be any therapeutic effect associated with the administration of a ubiquitin-proteasome system inhibitor. Therapeutic effects and/or diseases associated with the method include, but are not limited to, treatment of proliferative diseases, including but not limited to cancer, rheumatoid arthritis, autoimmune disease, transplant rejection, multiple sclerosis, sepsis, inflammatory bowel disease, lupus, proliferative disease, benign tumors, hyperplasias, and cachexia. In one embodiment, the therapeutic effect is the treatment of a cancer. The cancer can be selected from astrocytoma, adrenocortical carcinoma, appendix cancer, basal cell carcinoma, bile duct cancer, bladder cancer, bone cancer, brain cancer, brain stem glioma, breast cancer, cervical cancer, colon cancer, colorectal cancer, cutaneous T-cell lymphoma, ductal cancer, endometrial cancer, ependymoma, Ewing sarcoma, esophageal cancer, eye cancer, gallbladder cancer, gastric cancer, gastrointestinal cancer, germ cell tumor, glioma, hepatocellular cancer, histiocytosis, Hodgkin lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer and Wilms tumor. In some embodiments, the cancer is multiple myeloma.

### 5.4 COMBINATION THERAPY

One or more biologically active ubiquitin-proteasome system inhibitors are administered before, concurrently, or after administration of the micropinocytosis inhibitor. In one embodiment, the ubiquitin-proteasome system inhibitor is administered concurrently with the micropinocytosis inhibitor. In another embodiment, the ubiquitin-proteasome system inhibitor is administered before administration of the micropinocytosis inhibitor. When administered before the micropinocytosis inhibitor, the ubiquitin-proteasome system inhibitor is preferably administered 1, 2, 3, 4, 5, 6, 8, 10, 12, 18, 24, 30, or 45 minutes, hours, or days prior to the micropinocytosis inhibitor administration. In those embodiments that include bortezomib as a ubiquitin-proteasome system inhibitor, bortezomib can be dosed at 1.3 to 1.5 mg/m² of subject body surface on day 1, 4, 8, 11 and 21 and repeating such regimen after a two week interval.

It will be appreciated that the ubiquitin-proteasome system inhibitor and the micropinocytosis inhibitor can be administered as a pharmaceutically acceptable salt, ester or pro-drug thereof, or in a pharmaceutically acceptable carrier or diluent. The ubiquitin-proteasome system inhibitor and/or the micropinocytosis inhibitor described herein may be derivatized at functional groups to provide pro-drug derivatives that are capable of conversion back to the parent compounds in vivo.

Examples of suitable carriers or excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols. The ubiquitin-proteasome system inhibitor and micropinocytosis inhibitor may additionally be administered in compositions that include lubricating agents such as talc, magnesium stearate, and mineral oil; wetting agents; emulsifying agents; suspending agents; preserving agents such as methyl-, ethyl-, and propyl-hydroxy-benzoates; pH adjusting agents such as inorganic and organic acids and bases; sweetening agents; and flavoring agents.

Administration of the ubiquitin-proteasome inhibitor and micropinocytosis inhibitor with one or more suitable pharmaceutical excipients as advantageous are carried out via any of the accepted modes of administration and as the term “administering” is defined above. In one embodiment, the ubiquitin-proteasome system inhibitor and micropinocytosis inhibitor are administered orally. The ubiquitin-proteasome system inhibitor and micropinocytosis inhibitor can be administered once or repeatedly, e.g. at least 2, 3, 4, 5, 6, 7, 8, or more times, or by continuous infusion.

### 5.5 COMPOSITIONS

In one embodiment, this disclosure relates to a composition comprising a combination of compounds as described herein and a carrier. In another embodiment, this disclosure relates to a pharmaceutical composition comprising a combination of compounds as described herein and a pharmaceutically acceptable carrier. In another embodiment, this disclosure relates to a pharmaceutical composition comprising an effective amount or a therapeutically effective amount of a combination of compounds as described herein and a pharmaceutically acceptable carrier.

In some embodiments, the composition, including pharmaceutical compositions comprises a combination of a ubiquitin-proteasome system inhibitor, macropinocytosis inhibitor, and other therapeutic agents. Compositions contemplated by the present disclosure can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical composition and formulations described herein are administered to a subject by multiple administration routes, including but not limited to, parenteral, oral, buccal, rectal, sublingual, or transdermal administration routes in one aspect, to carry out the methods as disclosed herein. In some cases, parenteral administration comprise, or consists essentially of, or yet further consists of, s intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration. In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium cascinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. Sec, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pa. 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999). In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range. In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate. In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some embodiments, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some embodiments, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch, methylcellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as magnesium aluminum silicate, a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In one embodiment, the extended release composition includes microparticles having a diameter from about 1 μm to about 10 μm. The microparticles can have a diameter of 1 μm, 2 μm, 3 μm, 4 μm, 5 μm, 6 μm, 7 μm, 8 μm, 9 μm, or 10 μm. In another embodiment, the extended release compositions include nanoparticles have a diameter ranging from 10 nm to 950 nm. The nanoparticles can have a diameter of 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 950 nm.

In some embodiments, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like. Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, stearic acid, sodium stearates, glycerol, talc, waxes, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil, a surfactant, and the like. Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, tricthyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like. Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, BASF, and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof. Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art. Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives. Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art. For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients, which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

### 5.6 pH-RESPONSIVE DELIVERY SYSTEM

Provided is a nanoparticle comprising a pH-responsive polymer, a pH-insensitive polymer that delivers one or more compounds that are hydrophobic and/or hydrophilic. The nanoparticle can act as a system for delivery of the compounds that are hydrophobic and/or hydrophilic. In certain embodiments, the delivery system comprises nanoparticles that release the hydrophobic and hydrophilic compounds in a pH sensitive manner.

### 5.7 DOSING REGIMEN

In some embodiments, the compositions disclosed herein are administered to a subject in a therapeutically effective amount. As used herein the term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

For the disclosed compositions, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. For the disclosed compositions, generally dosage levels of 0.001 to 20 mg/kg of body weight daily are administered to mammals.

In various embodiments, the inhibitor is administered at about 0.001-1000 mg/m², greater than 1000 mg/m², or less than 0.001 mg/m². In various embodiments, the inhibitor is administered at about 0.001-1000 mg/kg, greater than 1000 mg/kg, or less than 1000 mg/kg. In various embodiments, the inhibitor is administered once, twice, three or more times. In various embodiments, the inhibitor is administered about once or more a day. In various embodiments, the inhibitor is administered for about 1 day or more.

In various embodiments, the effective amount of the inhibitor may include about 0.001-1000 μg/kg/day, greater than 1000 μg/kg/day, or less than 0.001 μg/kg/day. In various embodiments, the effective amount of the androgen deprivation therapy agent may include about 0.001 μg/m²/day, greater than 0.001 μg/m²/day, or less than 0.001 μg/m²/day. In various embodiments, the effective amount of the androgen deprivation therapy agent is any one or more of about 0.001-1000 mg/kg/day, greater than 1000 mg/kg/day, or less than 0.001 mg/kg/day. In various embodiments, the effective amount of the androgen deprivation therapy agent may include any one or more of about 0.001-1000 mg/m²/day, greater than 1000 mg/m²/day, or less than 0.001 mg/m²/day. Here, “mg/kg” refers to mg per kg body weight of the subject, and “mg/m²” refers to mg per m² body surface area of the subject.

In some embodiments, the inhibitor may be administered at the prevention stage of a condition (i.e., when the subject has not developed the condition but is likely to or in the process to develop the condition). In other embodiments, the inhibitor may be administered at the treatment stage of a condition (i.e., when the subject has already developed the condition).

### 5.8 KITS

One aspect of the present disclosure provides a kit comprising a composition of the present disclosure and instructions for use. In some embodiments, one or more compositions disclosed herein are contained in a kit. In some embodiments, the kit comprises, consists essentially of, or consists of the one or more compositions disclosed herein and instructions for their use.

As used herein, a kit or article of manufacture described herein include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

The following Examples further illustrate the findings of the present disclosure.

### 6. EXAMPLES

We evaluated the influence of starvation on UPS in cancer cells. We found amino acid starvation may activate UPS for internalized protein degradation to provide nutrients for cancer cell survival. As a result, starved cancer cells became more sensitive to the UPS inhibition. Therefore, UPS may serve as a novel target for cancer starvation therapy. Our results showed that proteasome inhibition enhanced protein internalization as a feedback, which may provide supplementary nutrients through autophagic degradation. We hypothesized that inhibiting protein internalization by macropinocytosis inhibitor could significantly exacerbate amino acid starvation. Therefore, we explored the potential of the combination therapy with a UPS inhibitor, bortezomib (BTZ), and a macropinocytosis inhibitor, 5-(n-ethyl-n-isopropyl)-amiloride (EIPA) in cancer treatment. To co-deliver both hydrophobic drug, BTZ, and hydrophilic drug, EIPA, a pH-responsive polymersome delivery system was developed to further enhance drug accumulation at tumor tissues and control drug release in cancer cells. In stark contrast with previous nanomedicines consuming glucose or blocking amino acid transporter, we, for the first time, developed a coordinative nanocarrier targeting protein catabolismas a promising strategy for starvation-based cancer therapy (FIG. 1).

### 6.1 Amino Acid-Mediated Starvation Activates UPS for Internalized Protein Degradation

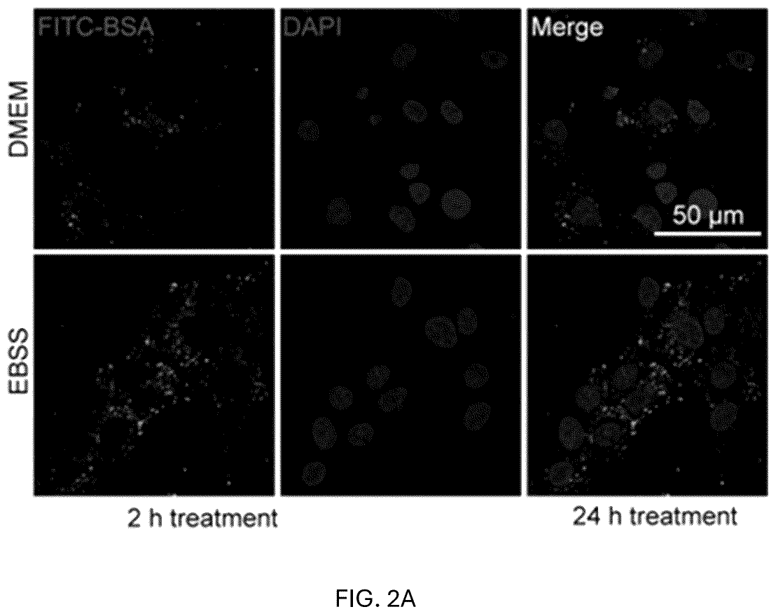

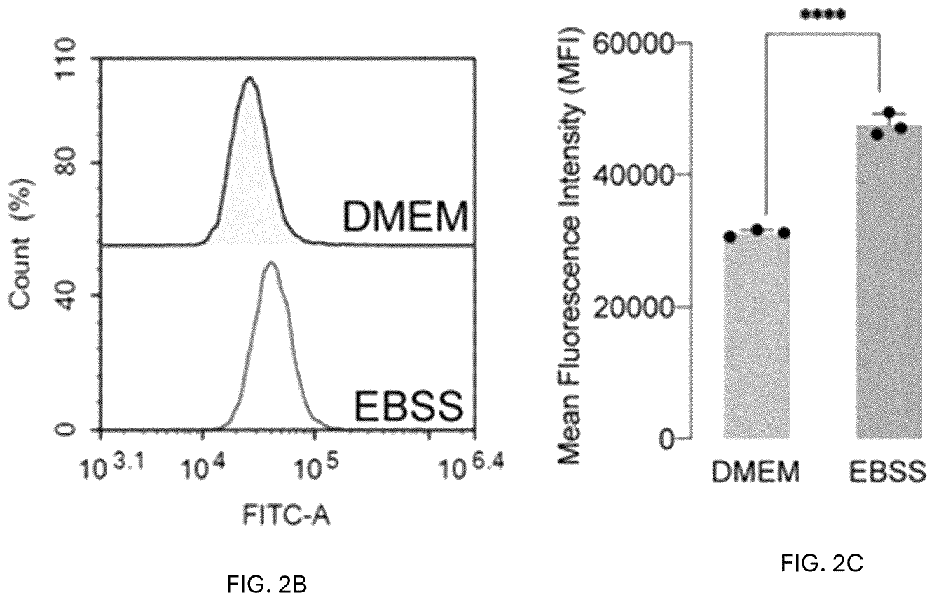

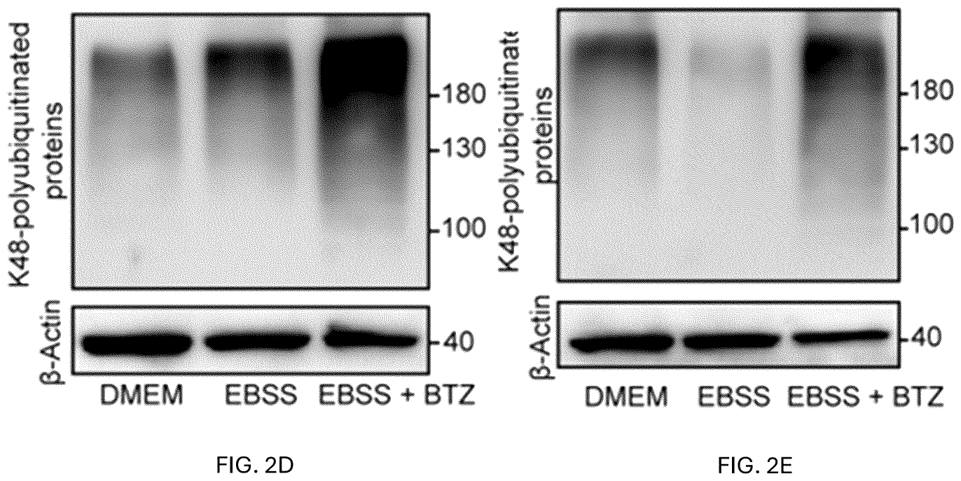

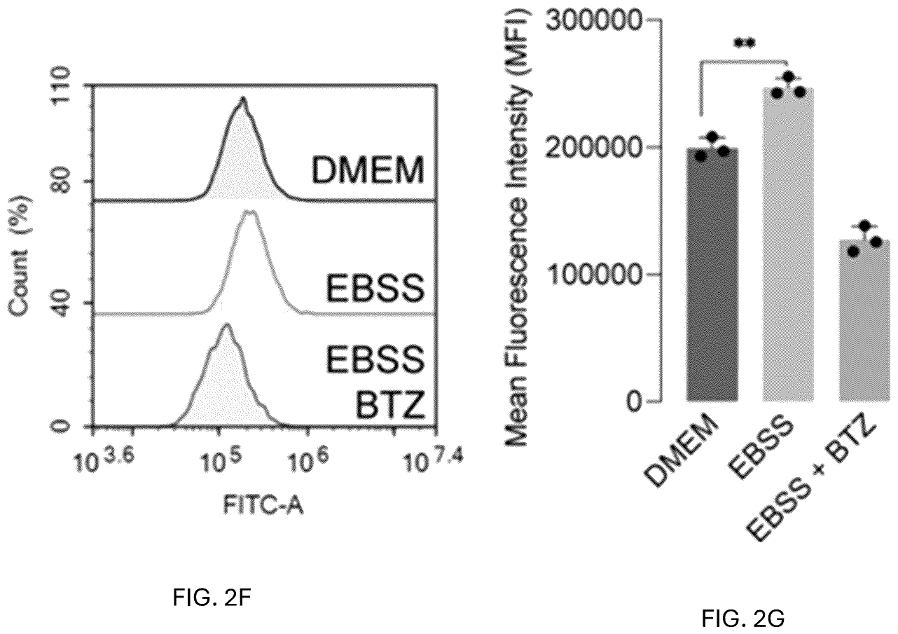

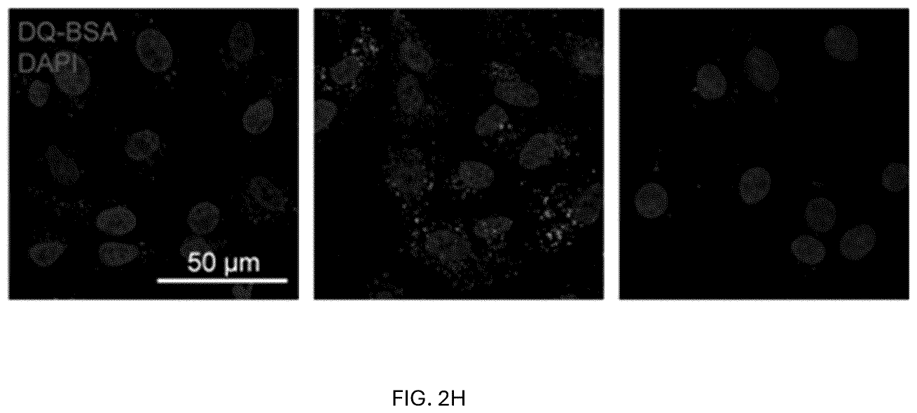

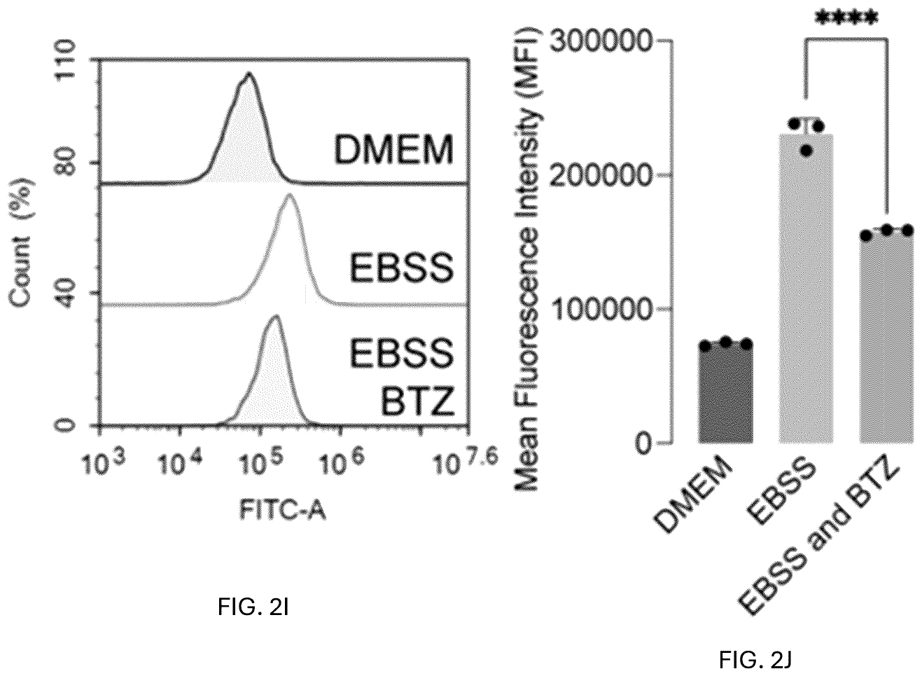

Firstly, we investigated the influence of amino acid-mediated starvation on protein internalization. To mimic amino acid starvation environment, human lung adenocarcinoma epithelial cells (A549) were cultured in Earle's balanced salt solution (EBSS) medium as a starvation model (2, 12). Fluorescein isothiocyanate-conjugated bovine serum albumin (FITC-BSA) was employed as a fluorescent biomarker to trace protein internalization. Compared with cells cultured in a normal condition, starved cancer cells displayed higher FITC-BSA internalization demonstrated by confocal laser scanning microscopy (CLSM) (FIG. 2A) and flow cytometry analysis (FIGS. 2, B and C). We further explored whether UPS could be activated in cancer cells subjected to amino acid starvation. For most proteins, ubiquitination is the rate-limiting step for protein breakdown by UPS (13), we therefore evaluated the level of K48-polyubiquitinated proteins after starvation, as K48-linked polyubiquitin chains are primarily involved in proteasomal degradation (14). Interestingly, in stark contrast with the cells maintained in Dulbecco's modified Eagle's medium (DMEM), short-term starvation (2 h) facilitated protein ubiquitination, which was notably decreased after long-term treatment (24 h), implying that depletion of amino acids facilitated protein ubiquitination at the early stage and promoted degradation of these polyubiquitinated proteins at the late stage (FIGS. 2, D and E). As an UPS inhibitor, BTZ led to profound accumulation of polyubiquitinated proteins both in short-term and long-term starved cancer cells, as it blocked protcasomal degradation (FIGS. 2, D and E). All these results indicated that amino acid starvation promoted protein ubiquitination and protcasomal degradation.

To further measure the UPS activity, a fluorescent proteasome probe (Mc4BodipyFL-Ahx3Lcu3VS) was utilized, which can specifically bind to the active site of proteasome and label it with green fluorescence. As indicated by flow cytometry, the fluorescence intensity of proteasome probe was elevated in amino acid starved cancer cells and remarkably reversed by BTZ treatment (FIGS. 2, F and G). Moreover, a constitutively fluorescent albumin (DQ-BSA) was applied to trace extracellular protein internalization and degradation process, which will only emit green fluorescence after albumin degradation. In starved cancer cells, the fluorescence intensity of DQ-BSA was increased compared with cells in normal medium and substantially decreased by BTZ, indicating that starvation facilitated internalized protein degradation in a UPS-dependent manner (FIG. 2, H-J).

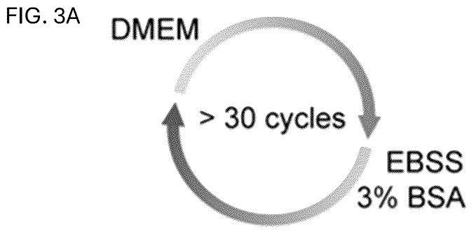

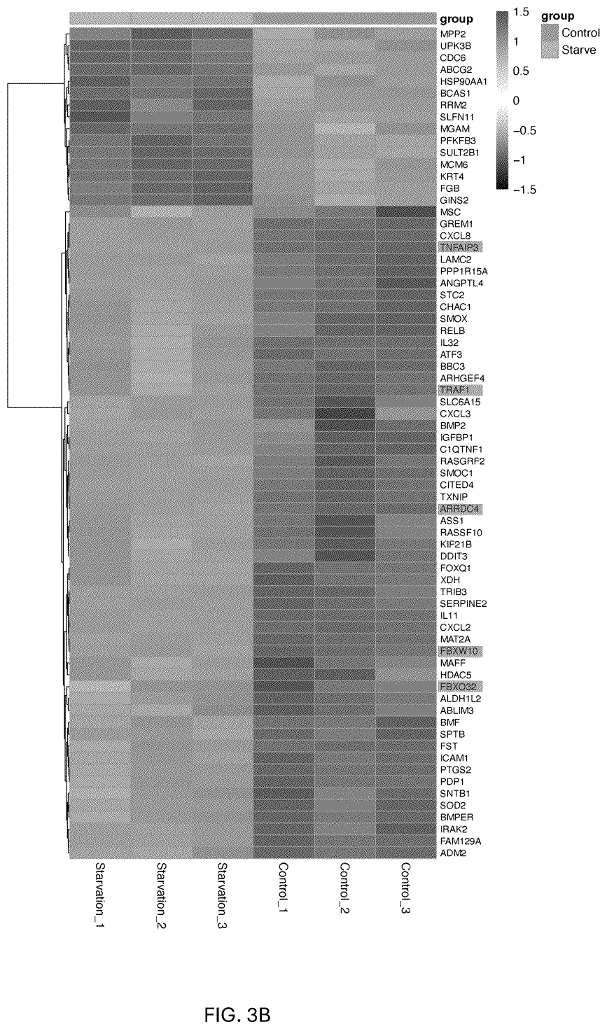

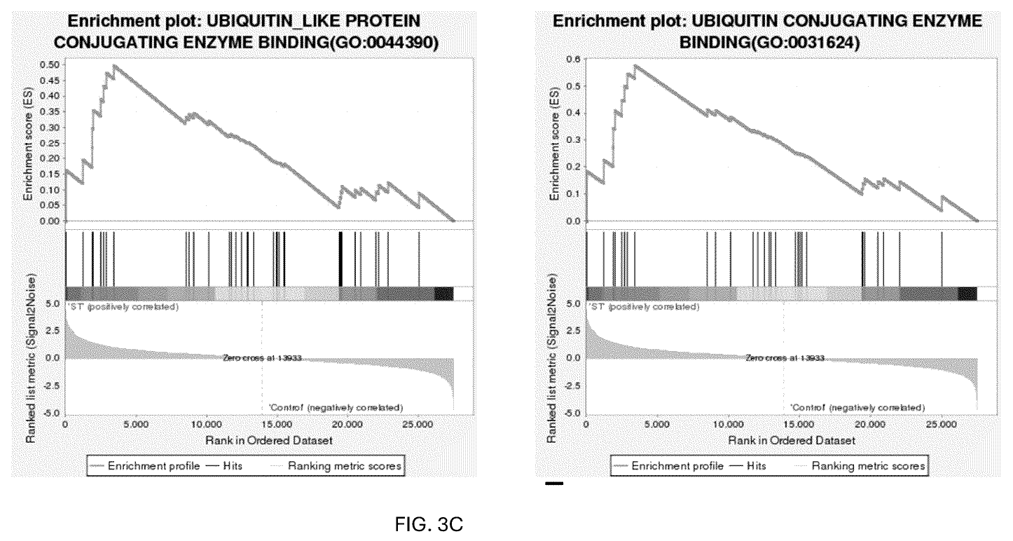

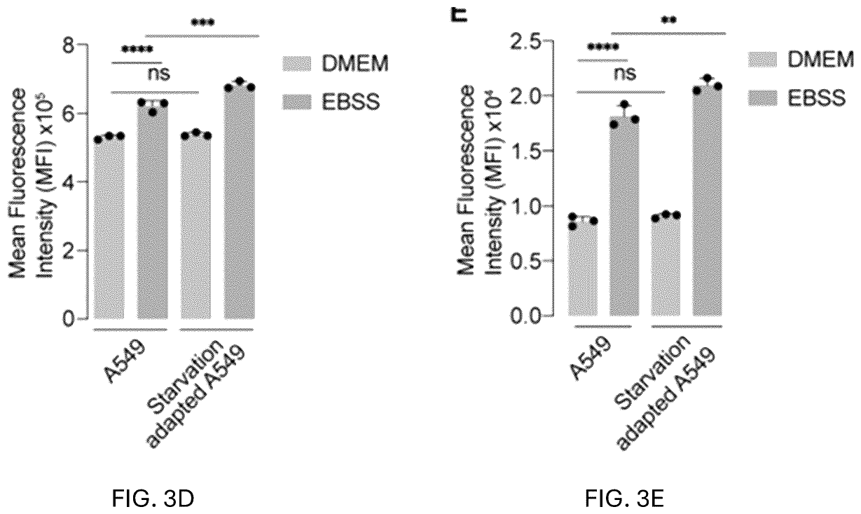

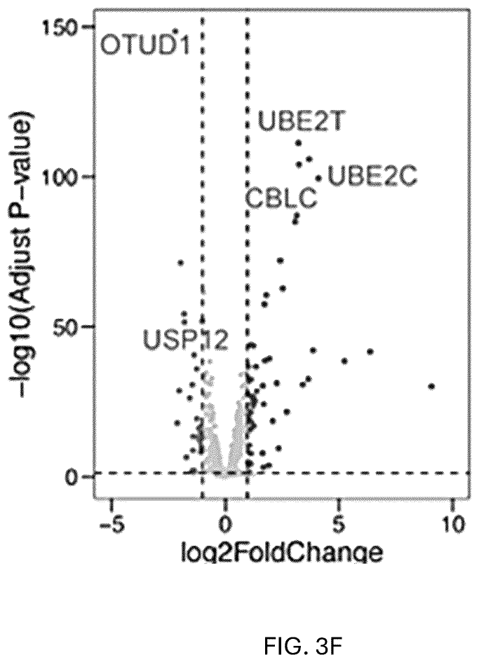

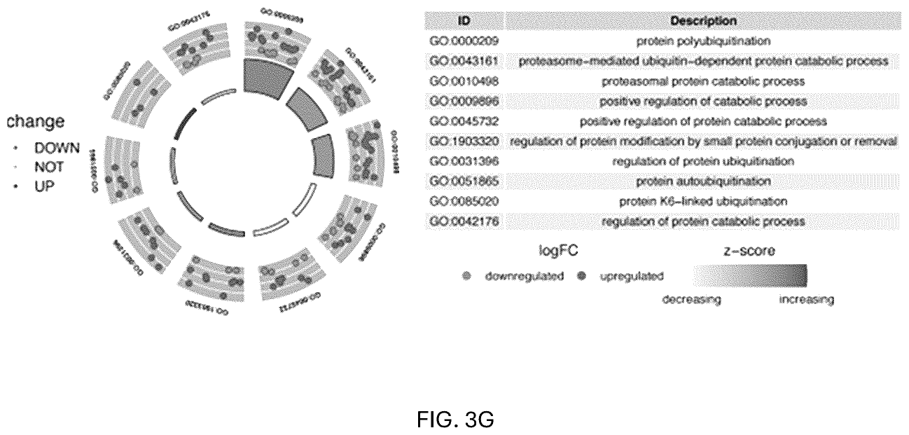

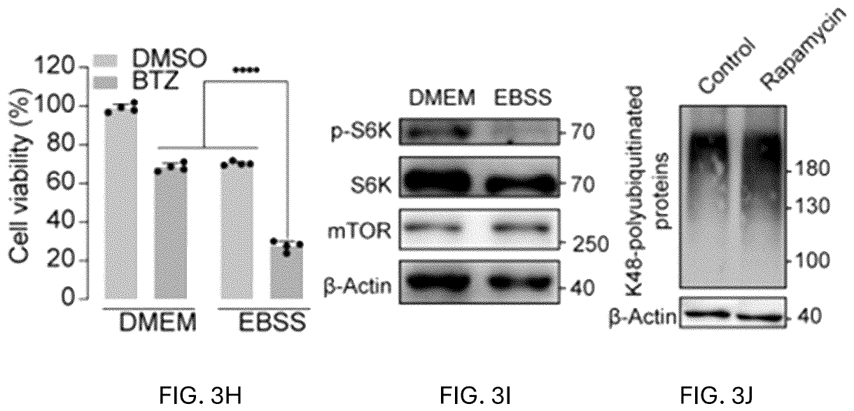

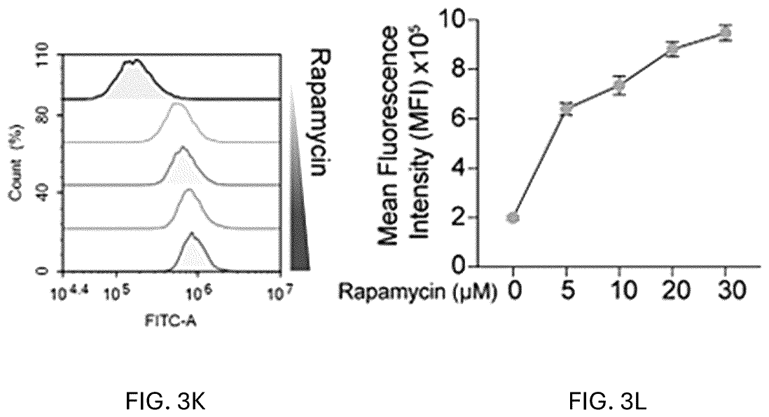

As solid tumors often develop into a chronic starvation microenvironment, we therefore investigated the UPS activity using a long-term, starvation-adapted cancer cell model (1). The cells were cultured in normal DMEM and EBSS (with 3% BSA) periodically for more than 30 generations (FIG. 3A). RNA sequencing (RNAseq) analysis was applied, and differentially expressed gene (DEG) analysis was performed between normal and starvation-adapted A549 cells. Among the top 70 DEGs, 5 UPS-related genes, including TNFAIP3, TRAF1, ARRDC4, FBXW10, and FBXO32, were upregulated in the starvation group. And all these five genes were positively associated with higher UPS activity (FIG. 3B). Gene set enrichment analysis (GSEA) was also investigated, suggesting that starvation was positively correlated with ubiquitin-like protein conjugating enzyme binding and ubiquitin conjugating enzyme binding activity (FIG. 3C). Intriguingly, when cultured in complete DMEM, starvation-adapted cells exhibited similar level of proteasome activity (FIG. 3D) and FITC-BSA uptake (FIG. 3E) compared with normal A549 cells. While under amino acid deprivation, starvation-adapted cells demonstrated higher proteasome activity (FIG. 3D) and FITC-BSA internalization (FIG. 3E).

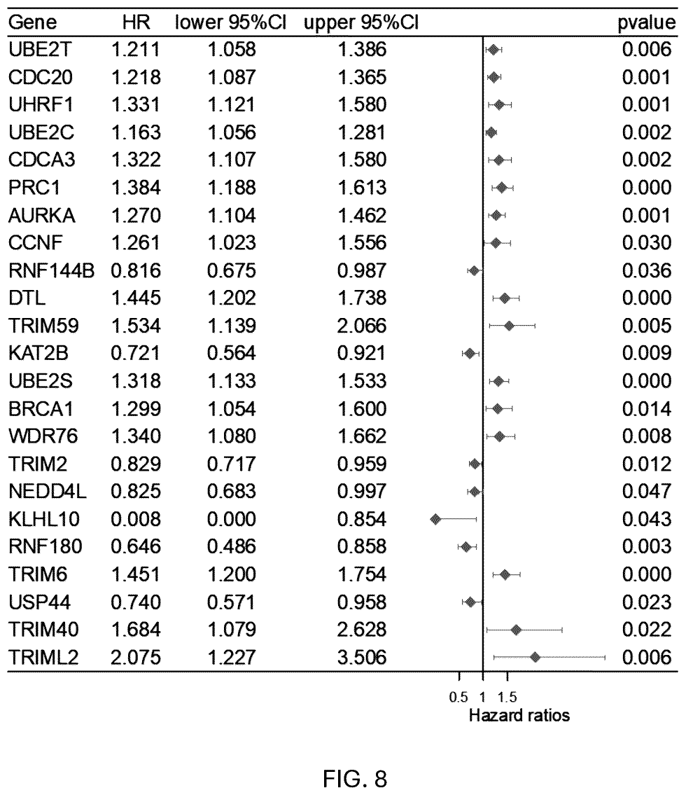

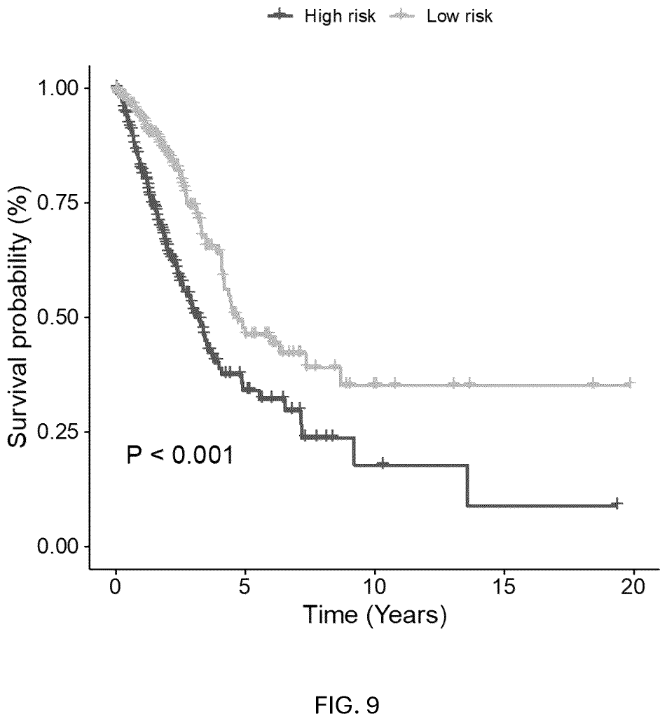

Considering high UPS activity elicited by amino acid starvation, we hypothesize that the UPS remains highly active in tumor tissues, which is more likely to be subjected to low nutrient supply in comparison to normal tissues (15). Therefore, we collected clinical RNAseq data from the cancer genome atlas (TCGA) database and compared DEGs between lung adenocarcinoma (LUAD) and normal tissues. As a result, genes encoding ubiquitin conjugating enzymes or ligases, such as UBE2T, UBE2C, and CBLC, were up-regulated in cancer patients, while genes encoding deubiquitinating enzymes like OTUD1 and USP12 were down-regulated (FIG. 3F). Gene ontology (GO) enrichment analysis also implied that lung cancer harnessed activated UPS-related pathways compared with normal tissues (FIG. 3G). Moreover, univariate Cox regression analysis was also investigated to identify that 23 genes among these 77 DEGs have statistical significance with LUAD patients' survival. And most of these genes were associated with poor prognosis in patients (FIG. 8). Using Lasso regression analysis, three genes including PRC1, TRIM6, and TRIML2 were found to act as independent prognostic signatures for patients. Higher expression of these three hallmark genes represented higher risk and lower survival rate in the patients (FIG. 9). As UPS activity was associated with prognosis in clinical patients, we investigated the anti-cancer effect of BTZ, especially in starved cancer cells. As a result, BTZ prompted a much more significant decrease of cell viability in starved cancer cells, in contrast to cells maintained in normal culture medium (FIG. 3H). To summarize, we found amino acid starvation induced UPS activation for extracellular protein degradation and rendered cells more vulnerable to UPS blockage. In clinical dataset, cancer lesion displayed up-regulation of UPS-promoting genes in contrast to normal tissues. And higher UPS-promoting gene expression was associated with poor prognosis in LUAD patients.

### 6.2 Starvation-Activated UPS is Mediated by mTOR Signaling Inhibition

mTOR signaling pathway is a critical node in coordinating protein and amino acid homeostasis. Sufficient intracellular amino acids activate mTORC1, which phosphorylates S6 kinase (S6K) and 4E binding protein (4E-BP) to initiate 5′ cap-dependent protein translation (16). Conversely, amino acid deprivation leads to mTORC1 inhibition and activates degradation of endocytosed proteins (2, 17). As mTORC1 activity constitutes a sensitive mechanism to monitor amino acids recovered from internalized proteins, we hypothesize that starvation-induced UPS activation maybe associated with mTOR signaling. A549 cancer cells were cultured in DMEM or EBSS medium for 24 h and S6K phosphorylation was evaluated using western blot analysis. According to the result, EBSS treatment led to lower S6K phosphorylation compared with the DMEM-treated group, indicating lower mTORC1 signaling activity (FIG. 3I). Rapamycin was employed as a mTORC1 inhibitor to further demonstrate the influence of mTORC1 activity on UPS level. According to the western blot result, the level of polyubiquitinated protein was elevated by rapamycin (FIG. 3J). Flow cytometry analysis was also employed to identify that rapamycin treatment enhanced proteasome activity in a dose-dependent manner (FIGS. 3, K and L). These results indicated that amino acid starvation can decrease mTORC1 activity, which can promote protein ubiquitination and elevate proteasome activity.

### 6.3 Synergistic Effect of UPS Inhibitor and Macropinocytosis Inhibitor

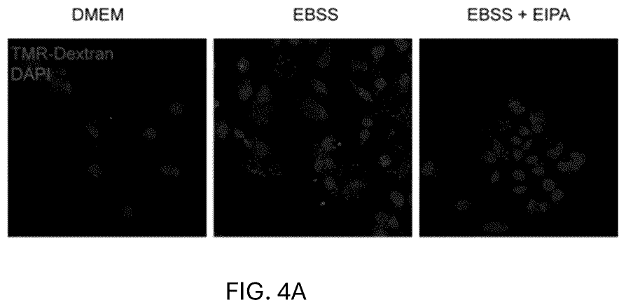

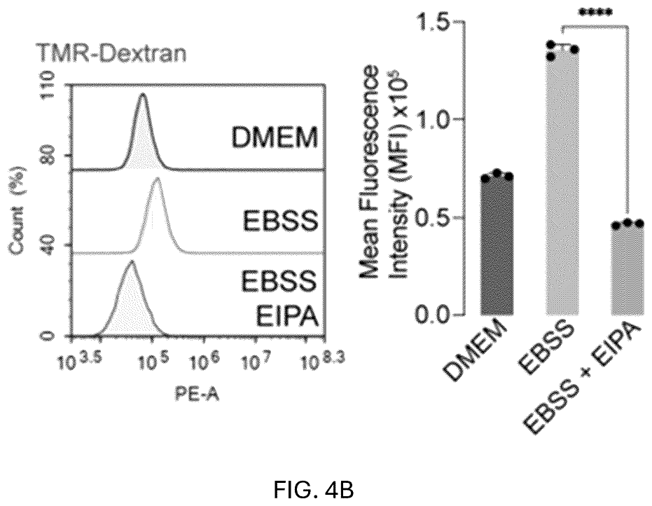

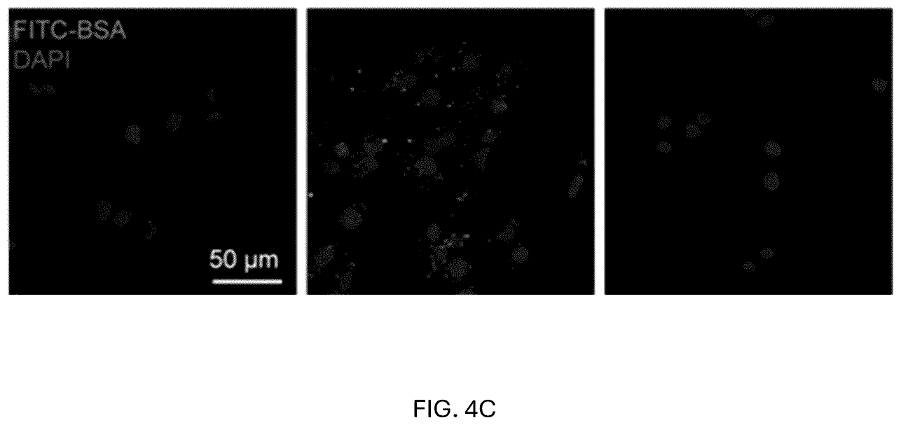

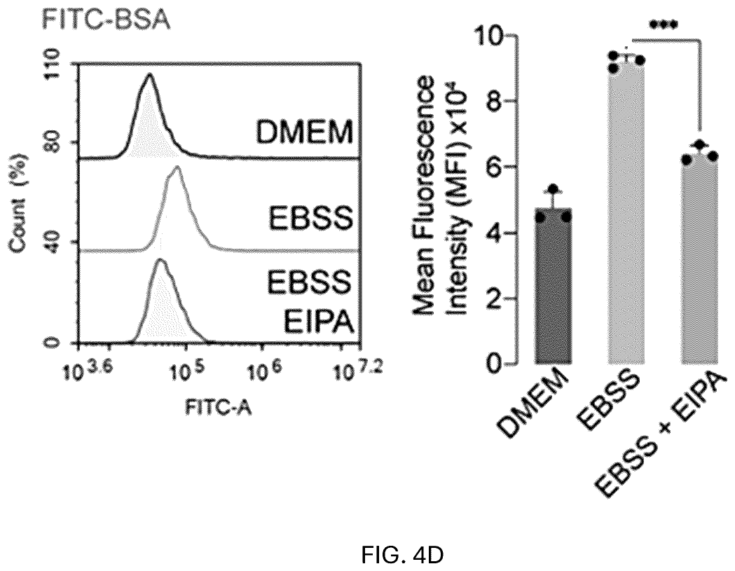

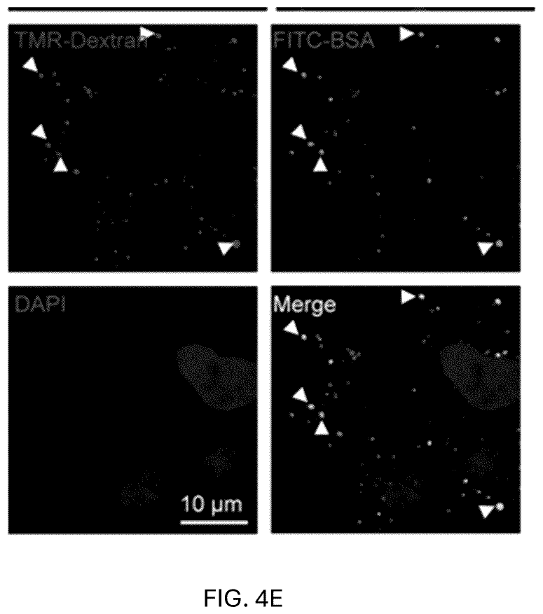

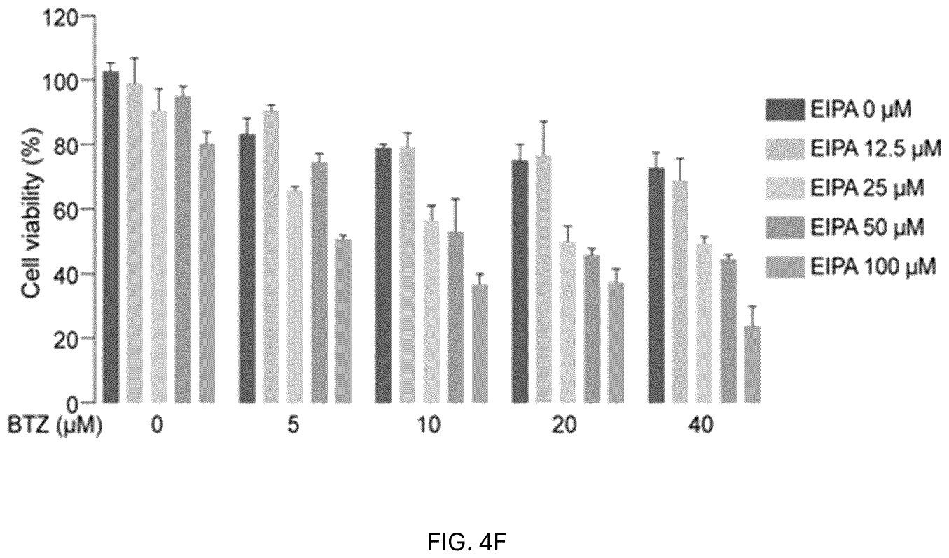

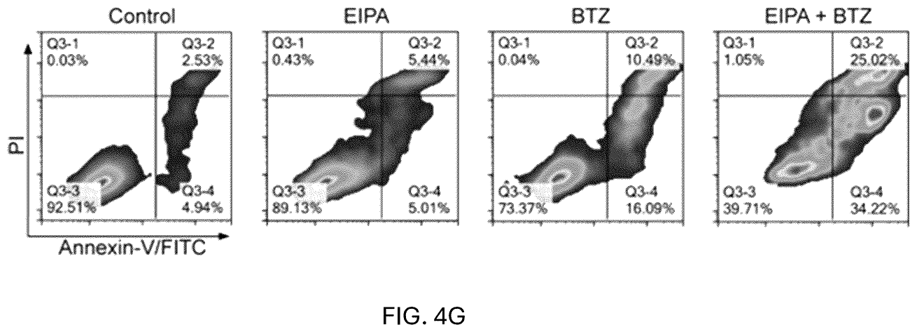

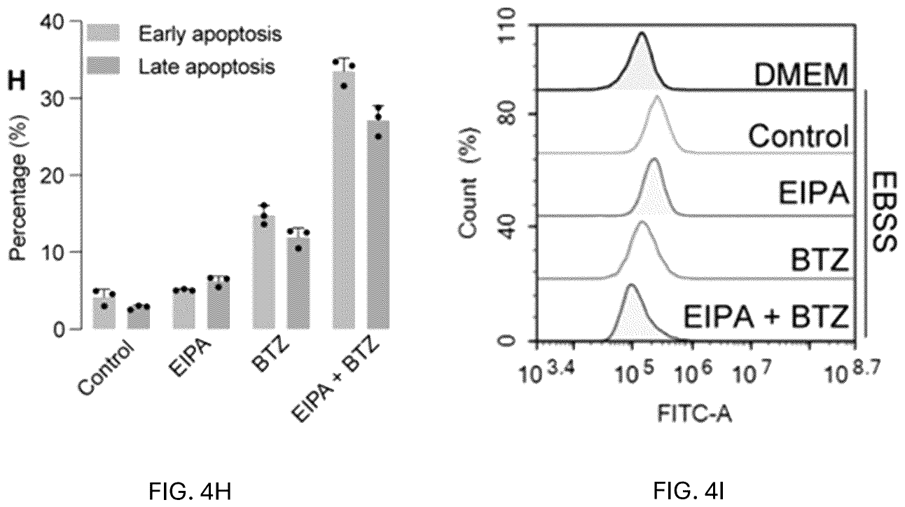

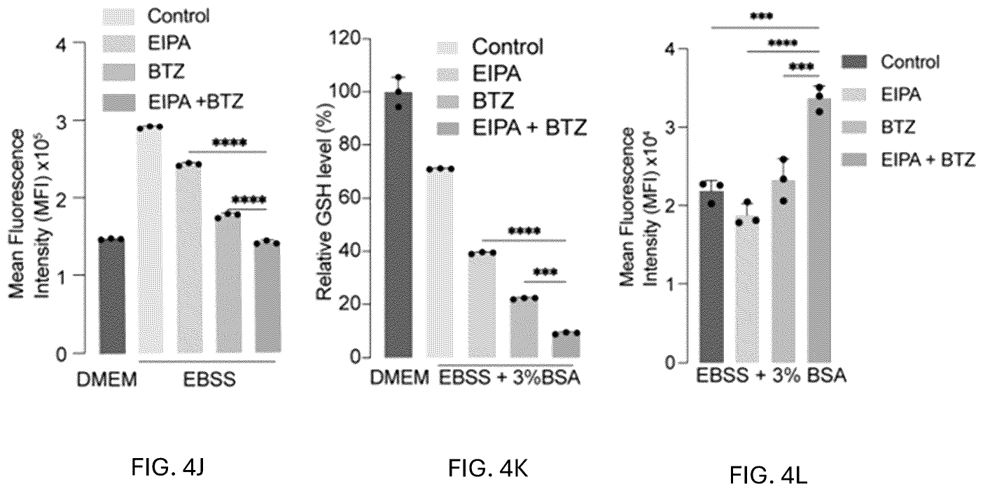

Based on the results mentioned above, BTZ has the ability to block UPS-dependent degradation of proteins in starved cancer cells, thereby limiting cell survival. However, extracellular proteins can still be internalized and degraded through lysosome-dependent degradation pathways (18, 19). Hence, the use of BTZ as a monotherapy for solid tumors may have limited anti-cancer effects (20). Therefore, we hypothesize that simultaneously blocking protein internalization and UPS-dependent degradation may further enhance the cancer starvation therapy. We focused on macropinocytosis pathway, because it plays an essential role in mediating protein internalization (4). Tetramethylrhodamine (TMR)-Dextran was utilized as a marker to validate that macropinocytosis was significantly enhanced by amino acid starvation and inhibited by EIPA (FIGS. 4, A and B). Through the CLSM analysis, the co-localization of FITC-BSA and TMR-Dextran was observed, suggesting that proteins can be internalized by macropinocytosis (FIG. 4E). Presumably, the starvation-amplified FITC-BSA uptake was notably blocked by EIPA treatment (FIGS. 4, C and D).

### 6.4 Cell Viability in Dual-Drug Strategy

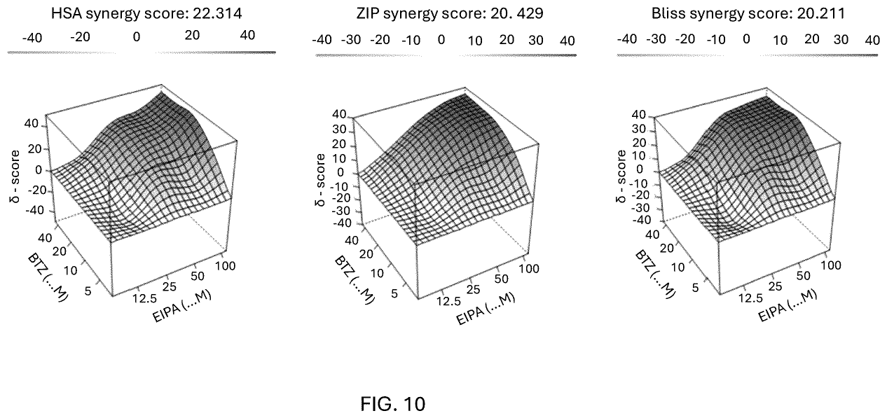

When we combined gradient concentrations of EIPA and BTZ together, the dual-drug strategy prompted a notable decrease of cell viability compared with each single treatment (FIG. 4F). The mean synergy scores calculated by SynergyFinder were higher than 20, implying that the cooperation of these two drugs was highly synergistic (FIG. 10) (21). According to the apoptosis analysis, the dual-drug strategy induced both higher early apoptosis (33.5%) and late apoptosis (27.1%) in contrast to each single treatment (FIGS. 4, G and H). The combination also facilitated notable decrease of DQ-BSA fluorescence intensity, attributed to the inhibition of protein internalization and UPS-dependent degradation (FIGS. 4, I and J). As amino acids, such as glutamate, cysteine and glycine, are requisite for glutathione (GSH) synthesis, the level of intracellular GSH may decrease as a result of reduced intracellular amino acid pool. Accordingly, the combination treatment induced a profound decrease of GSH (FIG. 4K), representing a liability to oxidative stress. The dual-drug combination led to the highest ROS generation evidenced by elevated 2′,7′-dichlorofluorescein diacetate (DCFH-DA) fluorescence intensity compared with other groups (FIG. 4L). Therefore, the decreased GSH level and elevated ROS generation may contribute to apoptotic cell death of the cancer cells.

### 6.5 Dual-Drug Combination Avoids Compensatory Amino Acid Supply

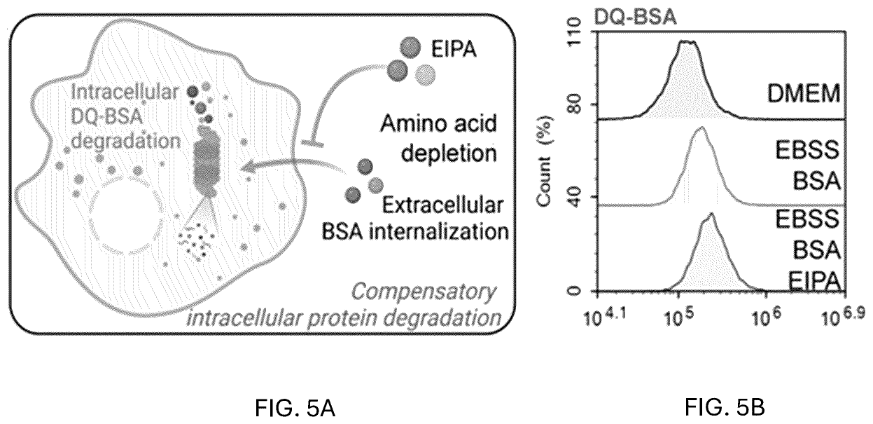

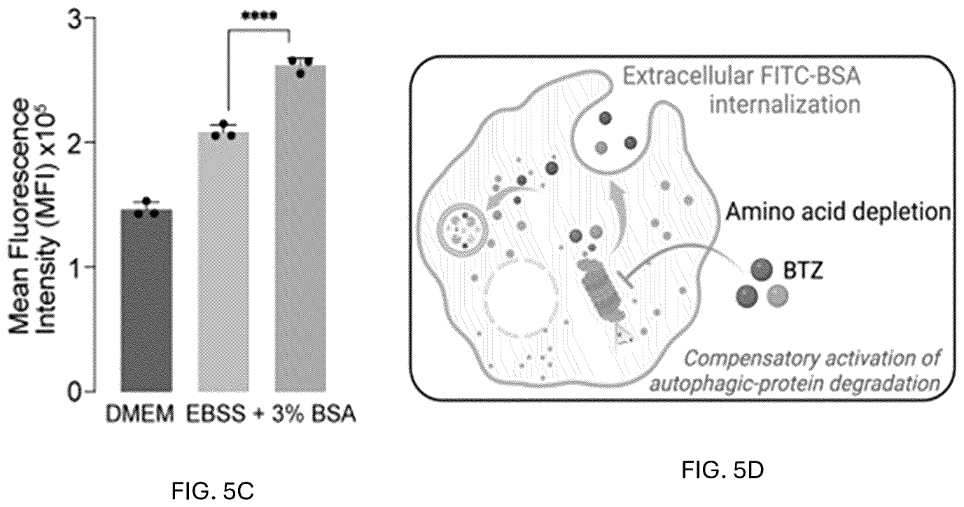

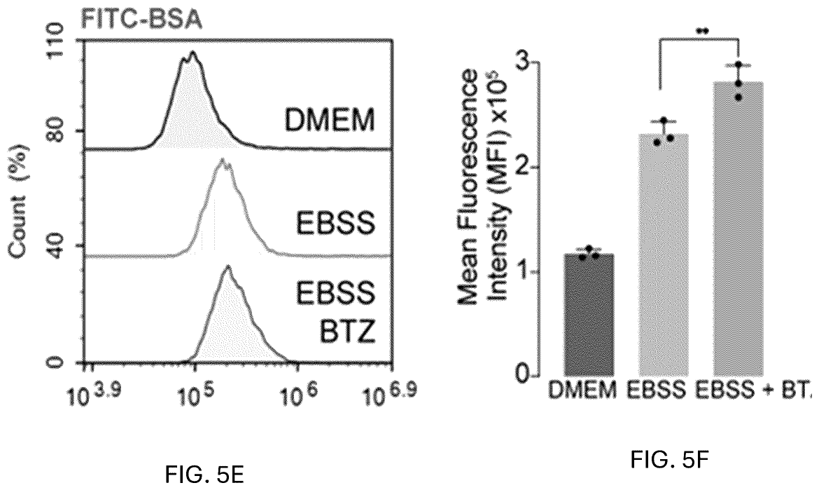

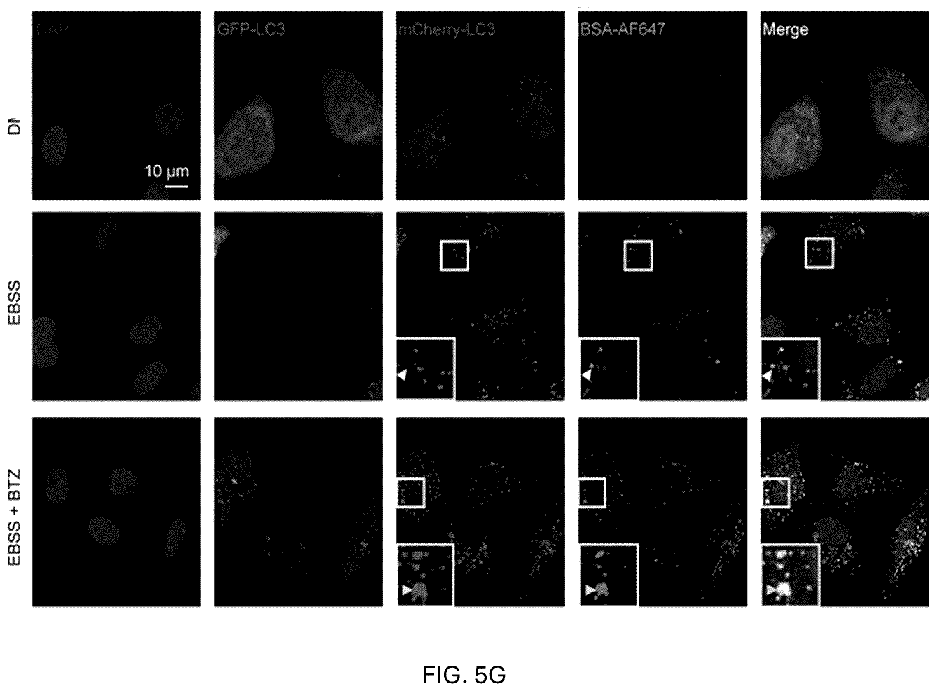

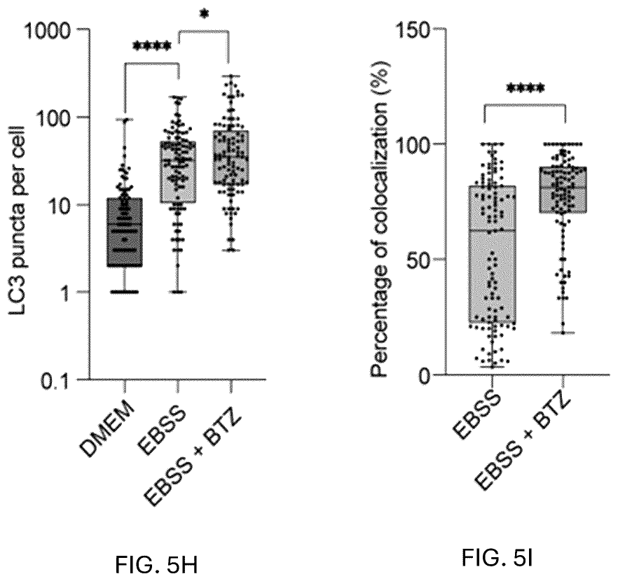

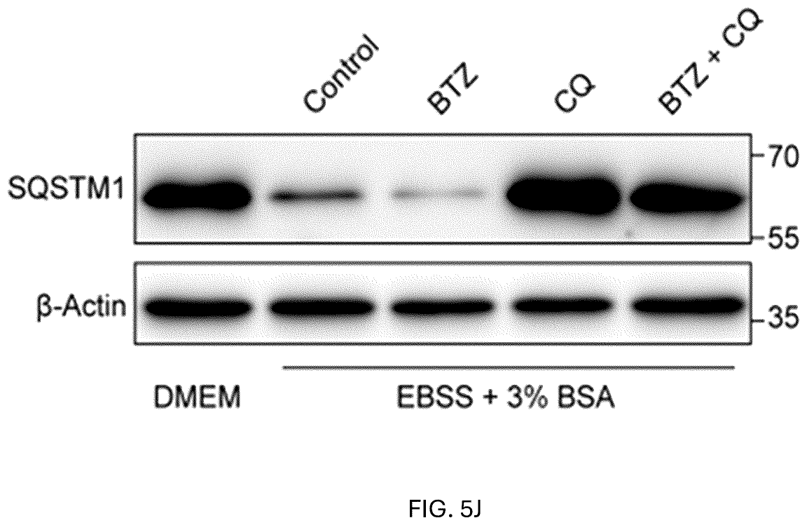

Besides phenotypically demonstrating the anti-cancer effect, we also investigated the detailed mechanism of the synergistic effect. We proposed that although EIPA as a single agent can inhibit extracellular protein internalization in amino acid-starved cancer cells, intracellular proteins may also be degraded to provide amino acids for cell survival. To define the influence of EIPA on intracellular protein degradation, we pre-incubated the cells with DQ-BSA for 1 h and changed the medium to DMEM, EBSS with 3% BSA and EBSS with 3% BSA plus EIPA, and incubated for another 3 h, respectively. Hence, the change of fluorescence intensity was induced by different treatments after DQ-BSA internalization, as a surrogate for intracellular protein degradation (FIG. 5A). According to flow cytometry analysis, EIPA further increased the fluorescence intensity of DQ-BSA compared with starvation group, suggesting that EIPA led to compensatory degradation of intracellular DQ-BSA through inhibiting unconjugated BSA uptake (FIGS. 5, B and C).

In addition, single BTZ treatment may not influence protein uptake, hence extracellular proteins may still be internalized and degraded through lysosome-dependent pathways (FIG. 5D). When the cells were incubated with FITC-BSA, treated with or without BTZ in DMEM or EBSS medium for 4 h, we observed that BTZ further increased protein internalization (FIGS. 5, E and F). We transduced A549 cancer cells with GFP-mCherry-LC3 plasmid to define the autophagy flux after different treatments. According to the CLSM analysis, we observed a notable increase of LC3 puncta in starved cancer cells compared with the control group, which was further enhanced by BTZ incubation (FIGS. 5, G and H). Moreover, compared with starved cancer cells, BTZ treatment increased the percentage of BSA-AF647 colocalized with LC3 puncta, implying that BTZ drove compensatory extracellular protein uptake and autophagy-dependent degradation (FIGS. 5, G and I).

As a chaperone protein for autophagy, SQSTM1 expression level was also investigated using western blot analysis. BTZ exacerbated starvation-induced SQSTM1 decrease, indicating that autophagy was amplified for protein degradation, which was further validated by turnover assay using chloroquine (CQ) as a late-autophagy inhibitor (FIG. 5J). To summarize, single treatment by EIPA or BTZ may contribute to activation of compensatory amino acid supply route to benefit cancer cell survival. Therefore, blocking protein internalization and UPS-dependent degradation simultaneously may provide a tentative strategy for cancer starvation therapy.

### 6.6 pH-Responsive Polymersome as the Dual-Drug Delivery System

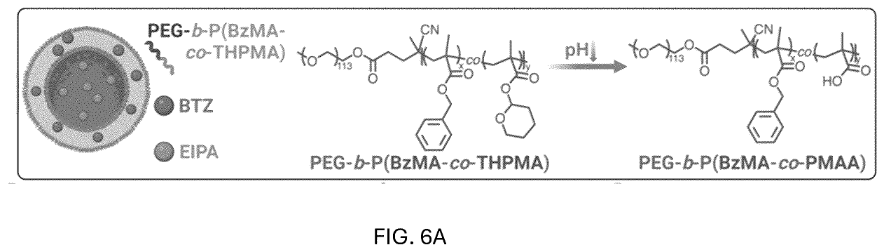

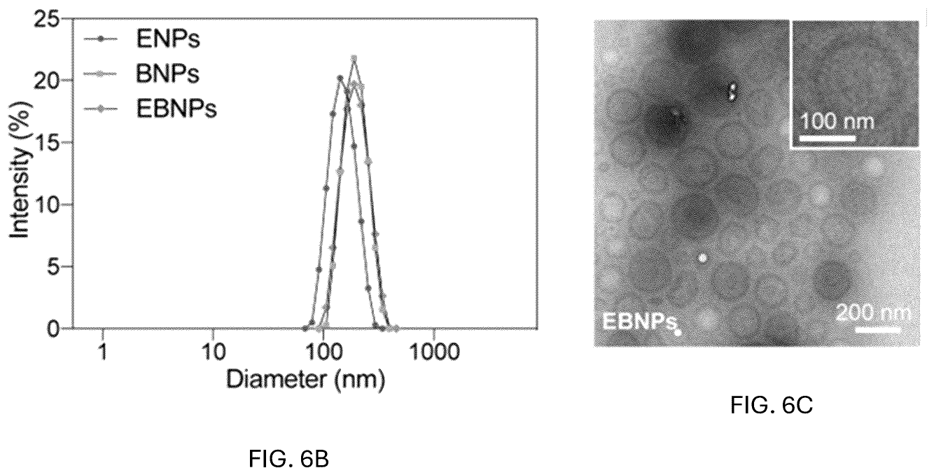

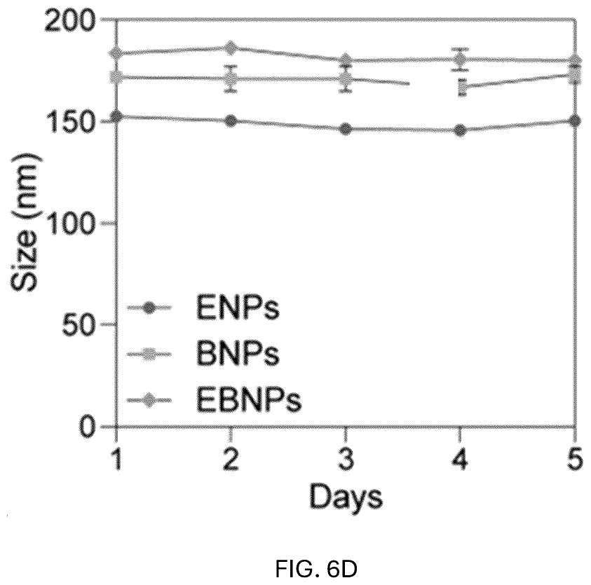

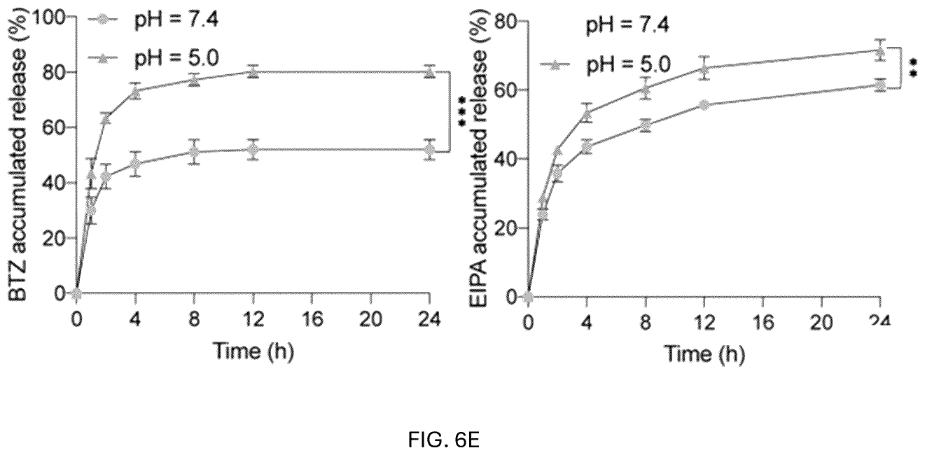

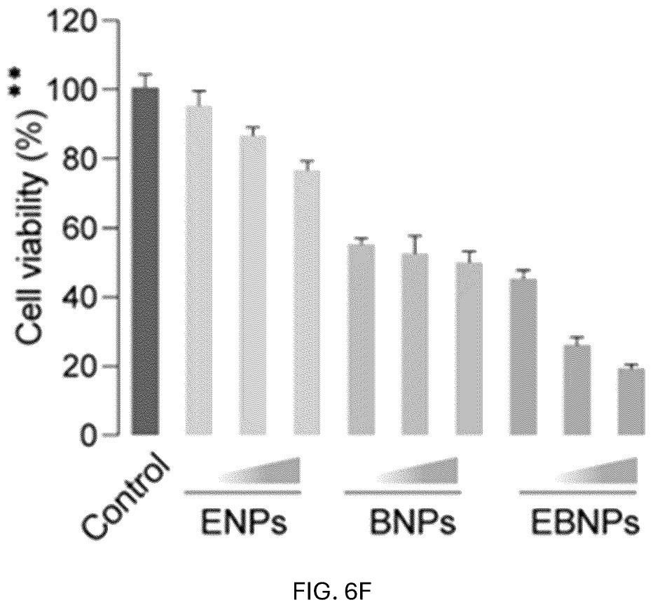

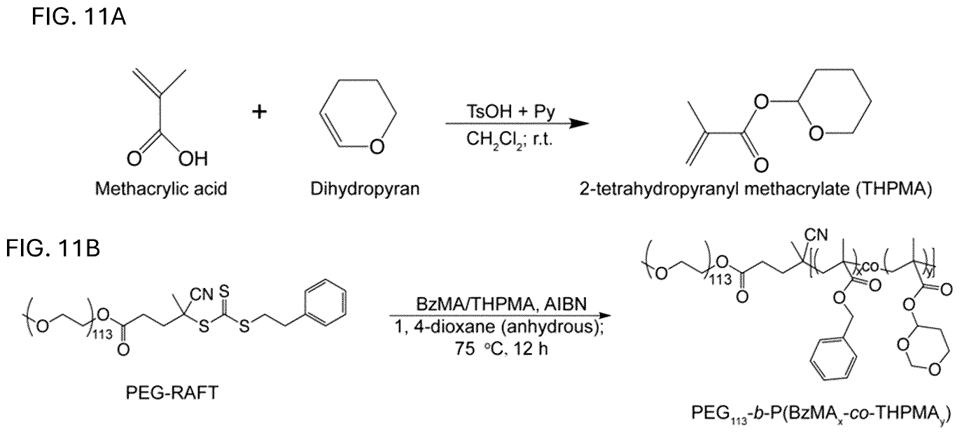

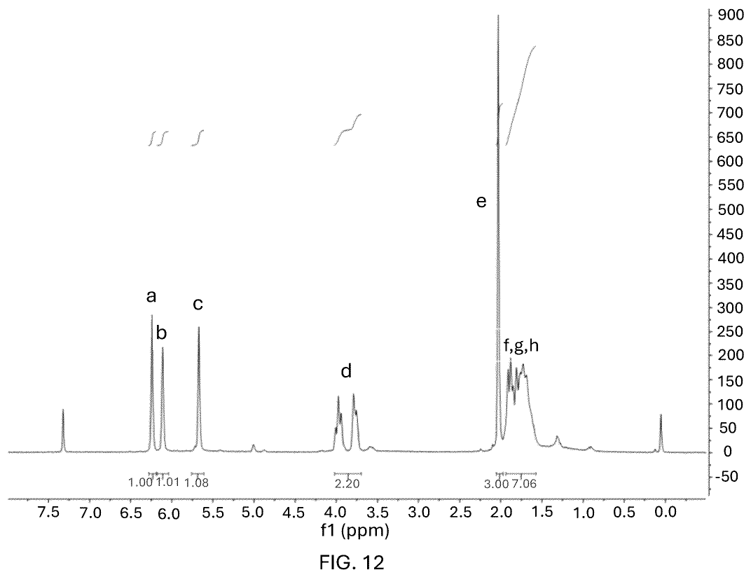

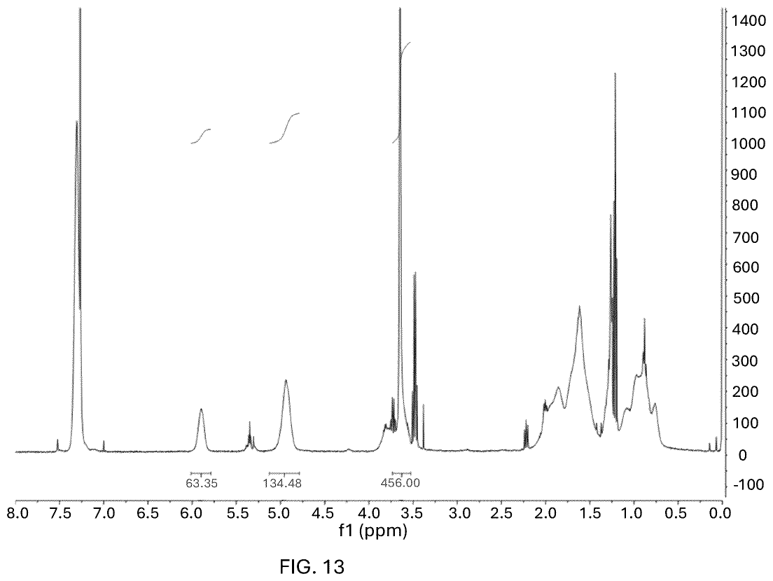

To further facilitate the clinical translation of this combination approach, nanodrug delivery strategy was applied in this study to enhance the drug accumulation at tumor sites while reducing unwanted adverse effects through enhanced permeability and retention (EPR) effect (22). A novel polymersome was developed to co-encapsulate EIPA into the hydrophilic core and BTZ in the hydrophobic membrane bilayer (FIG. 6A). It is noteworthy that a pH-responsive tetrahydropyranyl methacrylate (THPMA) moiety was incorporated to facilitate drug release in acidic environment, allowing specific drug release in the tumor tissues (FIG. 6A) (23, 24). The synthetic routes of the block copolymers were demonstrated in FIG. 11. The pH-responsive monomer, THPMA, was synthesized using methacrylic acid and dihydropyran (FIG. 12). The block copolymer, PEG-b-P(BzMA-co-THPMA), was synthesized via the reversible addition-fragmentation chain transfer (RAFT) copolymerization of BzMA and THPMA monomers using the PEG-RAFT agent. The BzMA and THPMA monomer polymerization degrees were determined to be 68 and 63, as confirmed by 1H-NMR analysis (FIG. 13). Hence, the synthesized block copolymer was referred as PEG₁₁₃-b-P(BzMA₆₈-co-THPMA₆₃).

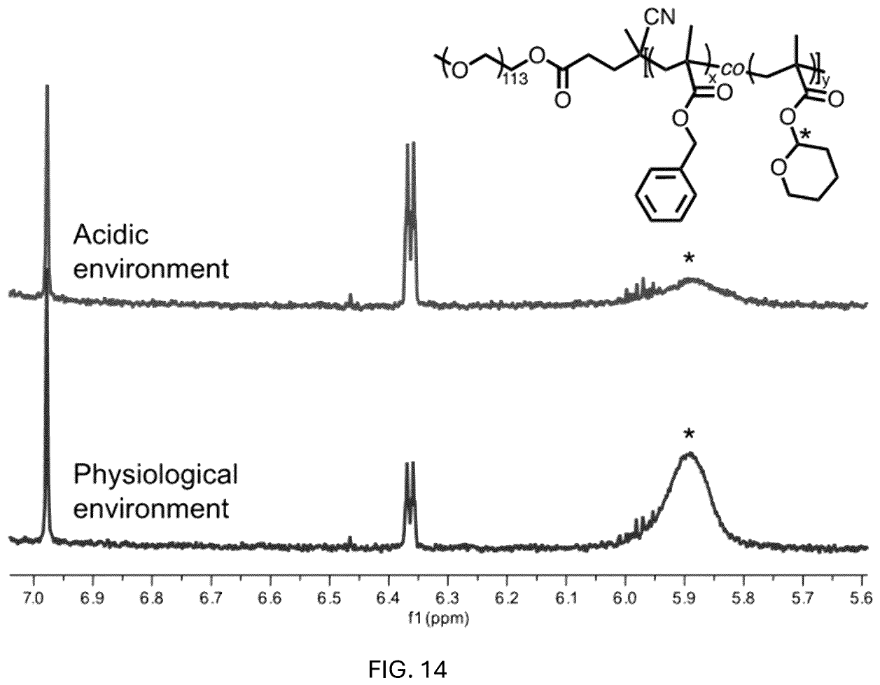

Subsequently, the copolymer was employed to form polymersomes to encapsulate EIPA and BTZ. According to the dynamic light scattering (DLS) measurement, the sizes of EIPA-loaded nanoparticles (ENPs), BTZ-loaded nanoparticles (BNPs), and EIPA and BTZ-loaded nanoparticles (EBNPs) were 143.5 nm, 188.9 nm, and 186.3 nm, respectively. And the polymer dispersity index (PDI) were all lower than 0.1 (FIG. 6B). These nanoparticle formulations remained stable in DMEM with 10% FBS at 37° C. for at least 5 days (FIG. 6C). 1H-NMR analysis was employed to characterize the pH responsiveness of this block copolymer (FIG. 14), demonstrating that the THPMA moiety can be cleaved in acidic environment. Drug release profile of the nanoparticles was characterized under pH 7.4 and pH 5.0, separately, using reversed-phase high-performance liquid chromatography (RP-HPLC). According to the result, the release of BTZ and EIPA was increased under acidic environment and reached 80.2% and 71.5% at 24 h, respectively, suggesting that these polymersomes may rapidly release drugs in acidic environment (FIG. 6D). We also incubated cancer cells with different formulations with gradient concentrations of drugs and validated that dual-drug nanoparticles exhibited the highest cell killing effect in vitro (FIG. 6E).

### 6.7 ANTI-CANCER EFFECT OF DUAL-DRUG NANOPARTICLES IN VIVO

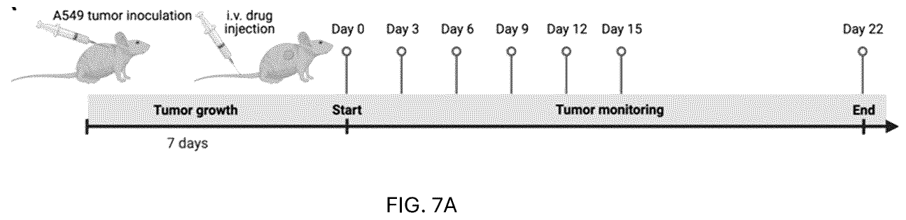

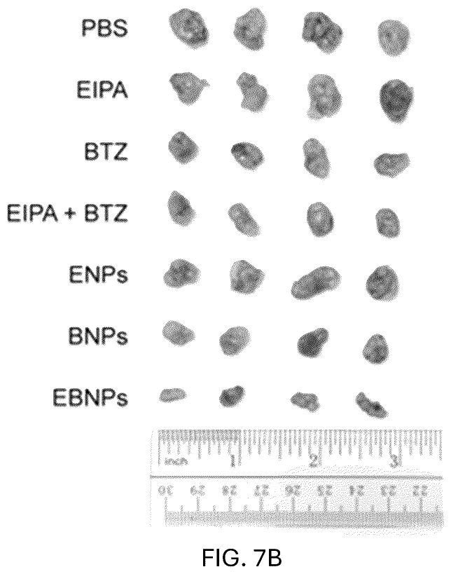

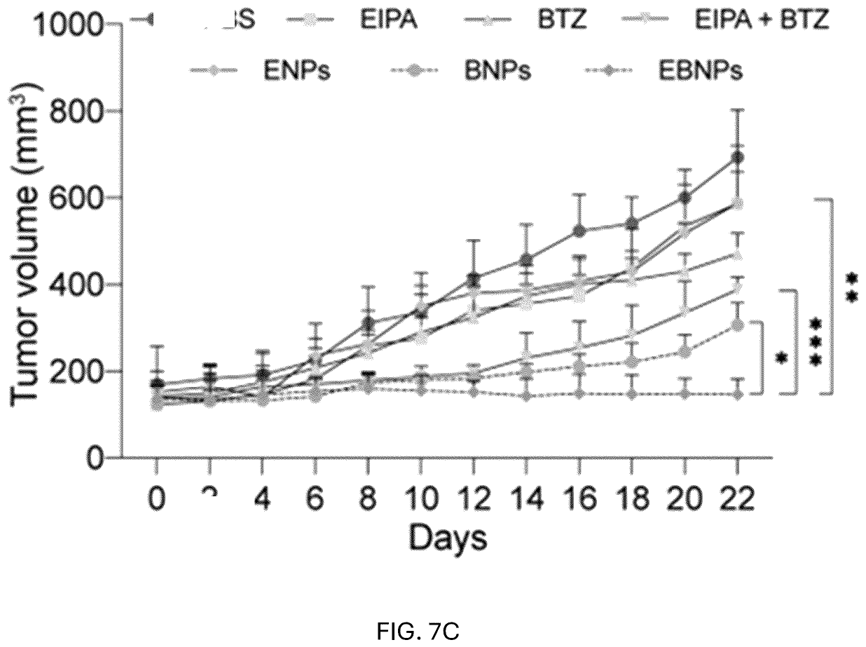

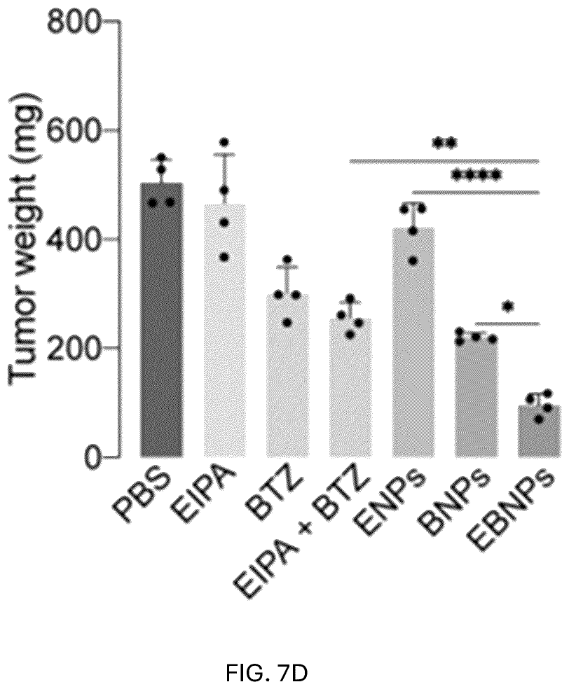

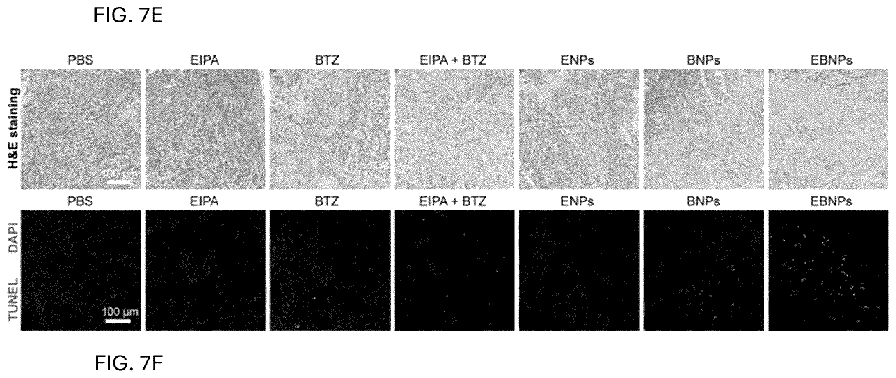

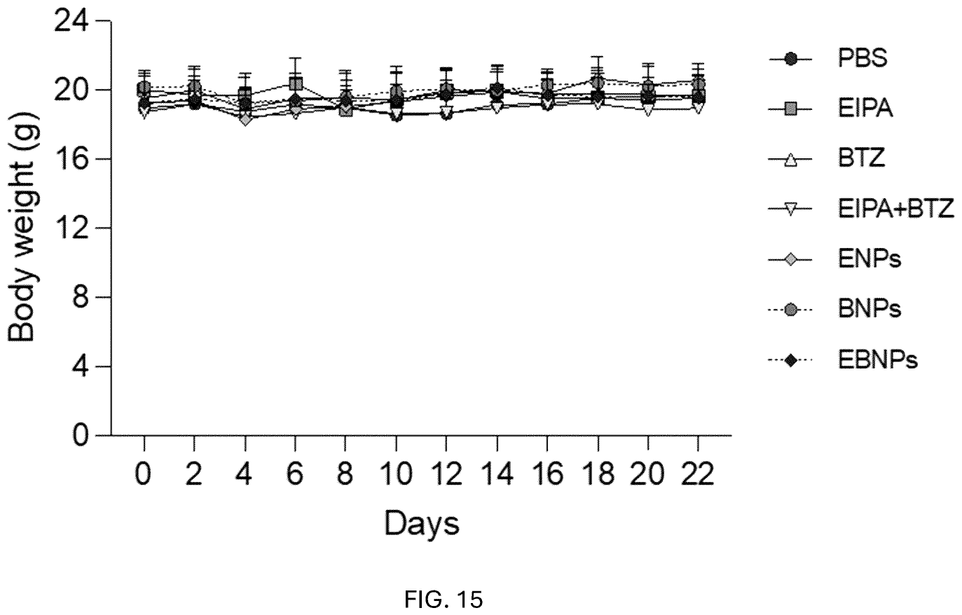

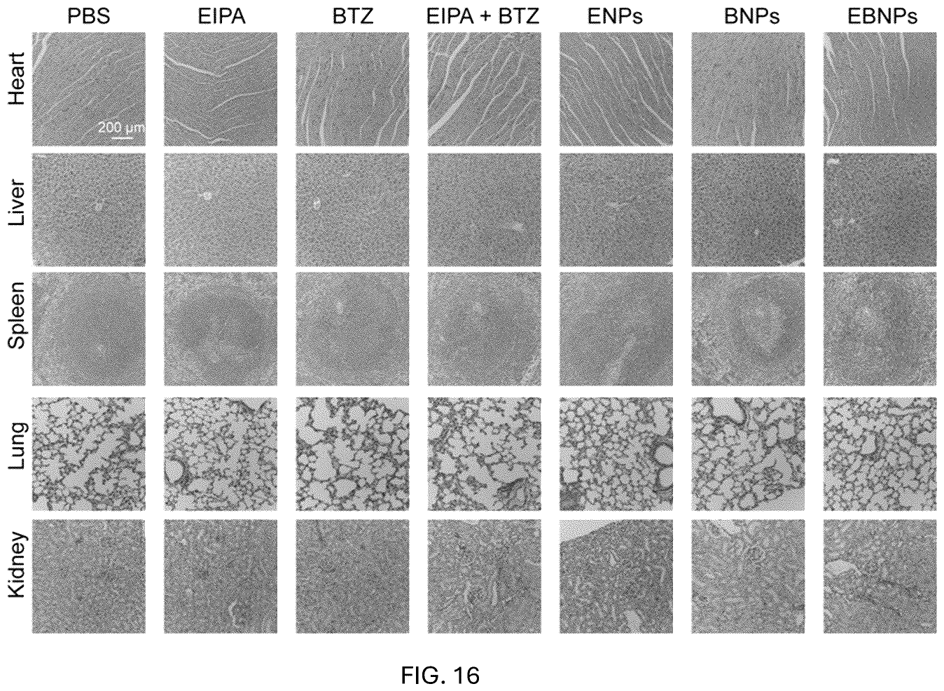

The applicability of the dual-drug polymersomes in vivo was also evaluated using xenograft mouse model. A549 cancer cells were inoculated at the right shoulder of the BALB/c nude mice. The tumor-bearing mice were divided into 7 groups, including PBS, EIPA, BTZ, EIPA+BTZ, ENPs, BNPs, and EBNPs. Different formulations (EIPA: 3 mg/kg, BTZ: 0.75 mg/kg) were intravenously injected into the mice six times at an interval of three days, separately (FIG. 7A). As dual-drug polymersome formulation, EBNPs exhibited significantly lower tumor volume and tumor weight compared with BNPs or ENPs (FIG. 7B-D), due to the combination effect of these two molecules. EBNPs also prompted higher anti-cancer effect in contrast to free-drug combination, possibly due to higher drug retention in the tumor tissues as nanoparticle formulation. Hematoxylin and eosin (H&E) staining analysis was performed in tumor tissues. In contrast to other groups, EBNPs treatment notably led to nuclear shrinkage and fragmentation with disappeared cell contour, suggesting desired ability to ablate tumor tissues (FIG. 7E). Terminal deoxynucleotidyl transferase dUTP nick end labeling (TUNEL) assay was also performed to evaluate apoptosis in tumor tissues. It was observed that EBNPs led to highest percentage of apoptotic cells in tumor tissues, in contrast to other groups (FIG. 7F). Biosafety profile was investigated to evaluate the biocompatibility of different formulations. Along the treatment procedure, all these groups did not show notable fluctuation of the body weight (FIG. 15). And there was no significant structural and morphological change in H&E staining of major organs including heart, liver, spleen, lung, and kidneys (FIG. 16), suggesting a desired biocompatibility of injected formulations used in this study.

### 6.8 DISCUSSION

Nutrient deprivation occurs in early stages of tumor development before formation of new blood vessels or in late stages owing to abnormal tumor vasculature (1, 3). Although this metabolic stress may restrict tumor growth, metabolically adapted cells could be selected due to chronic starvation, which may promote tumor progression (25). In starved cancer cells, extracellular proteins are engulfed through macropinocytosis pathway, degraded through lysosome-dependent pathway, and served as alternative resources to fuel amino acid pool (2, 26). As the other protein degradation pathway, UPS was traditionally assumed to serve distinct function with autophagy-lysosome system (18). Recent studies reported that starvation may also impact the UPS, while the results remained controversial. It was reported by Zhao et al. that amino acid starvation may enhance proteasome activity in HEK293 cells dependent on mTOR inhibition (18). While starvation may also elicit polyubiquitination of 26S proteasome and traffic it into autophagosome for autophagy-dependent degradation (27).

We defined that amino acid deprivation prompted high proteasome activity (FIGS. 2, F and G), thereby leading to augmented UPS-dependent internalized protein degradation in cancer cells (FIG. 2H-2J). A starvation-adapted cancer cell line was constructed to mimic chronically starved tumor microenvironment (12). Interestingly, this cell line adapted to long-term metabolic stress preferentially utilized amino acids in complete medium, while exhibited higher ability to internalize extracellular proteins for degradation (FIG. 3D, 3E). According to RNAseq analysis, the starvation-adapted cell line was characterized by up-regulation of UPS-promoting genes and activation of ubiquitination process (FIG. 3, A-C). In addition, based on clinical TCGA database, the UPS-related genes and pathways were also positively enriched in tumor tissues compared with normal tissues (FIGS. 3, F and G), associated with poor prognosis in LUAD patients (FIGS. 8 and 9). As starvation sensitized cancer cells to BTZ (FIG. 3H), UPS represented a potential target for cancer starvation therapy.

Before polyubiquitination and proteasomal degradation processes, extracellular proteins need to be internalized in cancer cells. It was observed in previous studies that macropinocytosis played a critical role in mediating extracellular protein uptake, which can be inhibited by macropinocytosis inhibitor, such as EIPA (4). And necrotic cell debris can also be internalized through macropinocytosis and provide nutrients for cell survival and rendered cancer cells resistant to chemotherapy or radiotherapy (28). Therefore, we hypothesize that blocking macropinocytosis and UPS-dependent degradation at the same time may exacerbate nutrient deprivation. As expected, combination of EIPA and BTZ displayed a synergistic cytotoxicity through promoting ROS generation (FIG. 4L) and apoptotic cell death (FIG. 4, F-H). This dual-drug strategy also decreased intracellular GSH level dramatically, demonstrating liability to oxidative stress (FIG. 4K). Intriguingly, while EIPA treatment alone can inhibit internalization of proteins (FIGS. 4, C and D), it also resulted in compensatory intracellular protein degradation (FIG. 5, A-C). Moreover, BTZ single treatment stimulated compensatory protein internalization, which can refuel amino acids by autophagic degradation (FIG. 5, D-J) (29). Through bypassing these mechanisms, the dual-drug combination strategy exhausted intracellular amino acids and prompted an exacerbated anti-cancer effect.

While these two small molecules were applied as a combination strategy to eliminate cancer cells, the clinical implementation could be further promoted by a nanodrug delivery system to co-deliver these two drugs, simplifying clinical administration (30). With prolonged circulation time, nanodrugs preferentially accumulate into tumor tissues due to permeable tumor vasculature and defective lymphatic drainage, known as EPR effect (31). And water solubility of EIPA and BTZ can also be increased to elevate effective concentration in blood circulation (32, 33). Therefore, a polymersome nanocarrier was developed to co-encapsulate these two drugs. It is noteworthy that previous nanoparticle-based approaches for cancer starvation therapy primarily focus on glucose consumption or amino acid transporter blockage, while we for the first time developed a coordination system targeting protein catabolism34, 35). According to the results, the pH-responsive THPMA moiety facilitated drug release in acidic environment (FIG. 6D) (23). The dual-drug polymersomes (EBNPs) exerted higher anti-cancer effect compared with free-drug administration or single-drug polymersomes (ENPs or BNPs) (FIG. 7, B-D). EBNPs also displayed significantly higher ability to ablate the tumors and elicit apoptosis in tumor tissues (FIGS. 7, E and F).

In conclusion, we found amino acid starvation can activate UPS for internalized protein degradation. Starved cancer cells displayed higher sensitivity to BTZ. Therefore, we combined BTZ with macropinocytosis inhibitor, EIPA, to block protein internalization and UPS-dependent degradation at the same time, to achieve synergistic anti-cancer effect by avoiding compensatory protein catabolism. To facilitate the clinical translation of this strategy, a pH-responsive polymersome was developed to co-deliver these two drugs, allowing enhanced drug retention in tumor tissues and higher therapeutic efficacy. By targeting protein catabolic process, this study provides a novel insight into cancer starvation therapy.

### 6.9 Materials and Methods

### 6.9.1 Materials

EIPA hydrochloride and BTZ were purchased from MedChemExpress LLC (New Jersey, USA). The proteasome probe, Me4BodipyFL-Ahx3Leu3VS, was purchased from R&D system (Minnesota, USA). Dimethyl sulfoxide (DMSO) was obtained from Sigma-Aldrich (Darmstadt, Germany). 3-(4,5-dimethyl-2-thiazolyl)-2,5-diphenyl-2-H-tetrazolium bromide (MTT) was obtained from J&K Co., Ltd (Beijing, China).

### 6.9.2 Cell Culture

A549 and 293 T cell lines were purchased from ATCC and used in this study. High glucose (4.5 g/mL) Dulbecco modified Eagle medium (DMEM, Gibco) containing 10% fetal bovine serum (FBS, Gibco) and 1% penicillin/streptomycin (Gibco) were used for cell culture. The cells were incubated in the incubator under 100% humidity, 5% CO2, 37° C. condition. For amino acid starvation, the cells were cultured in Earle's balanced salt solution (EBSS, Gibco), supplemented with glucose solution (Gibco) and MEM Vitamin solution (Gibco). To construct the starvation-adapted A549 cell line, the cells were incubated with EBSS (with 3% BSA) and complete DMEM periodically. The cells adapted to starvation were successfully constructed after 30 generations.

### 6.9.3 Fluorescent Imaging Analysis

For in vitro fluorescent imaging detection, the cells were seeded on Nunc glass bottom confocal dishes (Thermo Fischer Scientific, USA) for 24 h. Unless explained, the cells were incubated with FITC-BSA (0.2 mg/mL), BSA-AF647 (0.05 mg/mL), or DQ-BSA (10 μg/mL) for 4 h for protein internalization and extracellular protein degradation analysis, respectively. TMR-Dextran (0.125 mg/mL) was applied for 1 h as a macropinocytosis marker. Subsequently, after washed with PBS three times, the cells were fixed in 3.7% paraformaldehyde solution in PBS for 10 min, followed by washing with PBS three times and staining with DAPI according to the protocol (Thermo Fischer Scientific, D1306). After washing with PBS three times, the cells were mounted with SlowFade Diamond Antifade Mountant (Thermo Fischer Scientific, S36963). The in vivo TUNEL assay was conducted using the one-step TUNEL assay kit (C1088, Beyotime, China). The Zeiss LSM 900 inverted confocal microscope equipped with 10×0.45 NA, 20×0.8 NA and 40×1.4 NA lens was employed to analyze intracellular fluorescent signals. The same exposure settings were used across all conditions in each individual experiment.

### 6.9.4 Flow Cytometry Analysis

The cells were seeded in 12-well plates at 1×10⁵ density. Unless explained, the cells were incubated with FITC-BSA (0.2 mg/mL), DQ-BSA (10 μg/mL), or Me4BodipyFL-Ahx3Leu3VS (1 μM) for 4 h for protein internalization, extracellular protein degradation, and proteasome activity analysis, respectively. TMR-Dextran (0.125 mg/mL) was applied for 1 h as a macropinocytosis marker. After washing with PBS three times, the cells were harvested and analyzed by Agilent NovoCyte Quanteon analyzer.

### 6.9.5 Western Blot

The cells were plated in 12-well plates at 1×10⁵ density and incubated for 24 h. Subsequently, the cells received indicated treatments for 4 h and the cells were washed with 1×PBS three times and harvested with RIPA buffer (supplemented with Halt protease and phosphatase inhibitor cocktail, Thermo Fischer Scientific, USA). Total protein concentration was determined by pierce BCA protein assay kit (Thermo Fischer Scientific, #23225). Equal amounts of proteins were loaded. SDS-PAGE electrophoresis was utilized to separate proteins, which were subsequently transferred to a nitrocellulose membrane through the wet transfer system. The membranes were blocked with 5% BSA in TBST at room temperature for 1 h and incubated with indicated primary antibodies overnight under 4° C. After washed with TBST, the blots were incubated with secondary antibodies for 1 h at room temperature. After washed with TBST three times, the proteins blots were visualized by Clarity Western ECL Substrate (BioRad) and captured by ChemiDoc Imaging System (BioRad). The following antibodies were used: K48-polyubiquitinated proteins (Cell signaling technology, #8081), p-S6K (Cell signaling technology, #9234), S6K (Cell signaling technology, #2708), mTOR (Cell signaling technology, #2983), β-Actin (Cell signaling technology, #4967), SQSTM1 (Abcam, ab91526), HRP-conjugated secondary antibody (Abcam, ab6789), HRP-conjugated secondary antibody (Abcam, ab6721).

### 6.9.6 Cytotoxicity Assay

The cells were seeded in 96-well plates and cultured for 24 h. To investigate the influence of starvation on BTZ treatment, the cells were incubated with BTZ (20 μM) or DMSO, in complete DMEM or EBSS medium for 24 h. To investigate the combination effect of EIPA and BTZ, the cells were treated with a series of concentration of EIPA and BTZ in EBSS (plus 3% BSA) medium for 24 h. 10 μL of MTT (5 mg/mL) were added into each well and incubated for 2-4 h. Then the supernatant was changed with 100 μL DMSO to dissolve the precipitate. Subsequently, the absorbance of the solution at 570 nm and 630 nm was measured by a microplate reader (SpectraMax M4, Molecular Devices LLC, San Jose, CA). The cell viability was calculated by OD570-OD630 and normalized to DMSO control group.

### 6.9.7 Apoptosis Analysis

The cells were plated in 12-well plates at 1×10⁵ density and incubated for 24 h. Subsequently, the cells were incubated with EIPA (50 μM) or BTZ (20 μM), in EBSS (plus 3% BSA) medium for 24 h. Then the cells were washed with PBS three times, collected, and stained with Annexin-V/PI double staining kit (Beyotime, C1062S) according to protocol. Then the apoptosis was analyzed by Agilent NovoCyte Quanteon machine.

### 6.9.8 ROS Generation Analysis

The cells were seeded in 12-well plates at 1×10⁵ density for 24 h and treated with DMSO or indicated drugs in EBSS medium with 3% BSA for 24 h. Subsequently, the cells were incubated with DCFH-DA probe (10 μM) for 30 min. Then the cells were washed with PBS three times and harvested for flow cytometry analysis and the mean fluorescence intensity of DCFH-DA was measured by Agilent NovoCyte Quanteon system.

### 6.9.9 Glutathione (GSH) Level Analysis

The cells were seeded in 6-well plates at 1.5×10⁵ density for 24 h and treated with DMSO or indicated drugs in EBSS medium with 3% BSA for 24 h. Then the cells were washed with 1×PBS and harvested for detection. Intracellular GSH level was analyzed according to the protocol of Glutathione Colorimetric Detection Kit (Invitrogen, EIAGSHC). Subsequently, the absorbance of the solution at 405 nm was measured by the microplate reader.

6.9.10 mCherry-GFP-LC3 Transduction

The plasmid, pCDH-CMV-mC-G-LC3B-P, was a gift from Kazuhiro Oka (Addgeneplasmid #124974; http://n2t.net/addgene: 124974; RRID: Addgene_124974). psPAX2 was a gift from Didier Trono (Addgene plasmid #12260; http://n2t.net/addgene: 12260; RRID: Addgene_12260). pMD2.G was a gift from Didier Trono (Addgene plasmid #12259; http://n2t.net/addgene: 12259; RRID: Addgene_12259). The mCherry-GFP-LC3 transduced A549 was constructed according to our previous publication (1). Generally, the mCherry-GFP-LC3 lentivirus was produced by transduction in 293T cells using lipofectamine 2000 (Invitrogen, USA) with helper plasmids psPAX2 and pMD2.G. Sixteen hours after transduction, the cell culture medium was changed to complete DMEM medium. The supernatant was collected at 48 and 72 h after transduction, filtered through a 0.45 μm low-binding filter, and frozen at −80° C. until use. For lentiviral transduction, A549 cells were seeded at 60-70% density and incubated with the mCherry-GFP-LC3 lentivirus supernatant with polybrene. Positive cells were selected using 1 μg/mL puromycin (GoldBio) for 2 weeks.

### 6.9.11 Synthesis of Block Copolymer

The synthetic routes of pH-responsive copolymer was depicted in FIG. 11. Firstly, the monomer, 2-tetrahydropyranyl methacrylate (THPMA), was synthesized by acid-catalyzed esterification of Methacrylic acid with dihydropyran. Briefly, Methacrylic acid (4.0 g, 41.5 mmol), p-toluenesulfonic acid (0.35 g, 1.85 mmol) and pyridine (0.1467 g, 1.85 mmol) was dissolved in 35 mL of anhydrous dichloromethane. Dihydropyran (6.815 g, 81 mmol) was added into above solution dropwise, followed by stirring 24 h at room temperature. Subsequently, the reaction was terminated, and the solution was diluted with ethyl acetate and washed by brine. Finally, the organic solvent was collected and purified by column chromatography using ethyl acetate and hexane (1:6, v/v) as eluent to give pure THPMA monomer (5.15 g, yield: 66%).

Next, the amphiphilic pH-responsive copolymer was synthesized via RAFT copolymerization of BzMA and THPMA monomers using PEG-RAFT agent. Typically, PEG-RAFT agent (26 mg, 0.005 mmol), 2,2′-azobis(2-methylpropionitrile) (0.115 mg, 0.0007 mmol), BzMA monomer (71 mg, 0.4 mmol), THPMA monomer (68 mg, 0.4 mmol), and 1,4-dioxane (1 mL) were charged into a 5 mL Schlenk flask, evacuated by three freeze-pump-thaw cycles, and scaled under vacuum. The flask was placed in oil bath at 75° C. and the reaction was allowed for 18 h. Then the reaction solution was immersed into liquid nitrogen to terminate the reaction and added dropwise into cold diethyl ether for precipitation. Then the precipitate was centrifuged and collected. After repeating the dissolution-precipitation process twice, the product was dried under vacuum to yield the white product (77 mg).

### 6.9.12 Nanoparticle Preparation and Characterization

For the preparation of polysome nanoparticles, 2 mg polymers and 2 mg BTZ were dissolved in 1 mL organic solution (THF:DMSO=4:1). And 2 mg EIPA hydrochloride was dissolved in 1 mL ddH₂O. The EIPA solution was added into the stirring organic solution dropwise in 1 h using syringe pump (Longer Precision Pump Co., Ltd., China). Then 3 mL ddH₂O was added into the stirring solution in 3 h. Unencapsulated drugs were removed by ultrafiltration. The size and polydispersity index of the nanoparticles were measured by dynamic light scattering instrument (ZS90, Malven Instrument, Southborough, MA, USA). The nanoparticles were also dissolved in DMEM (with 10% FBS) for stability test for 5 days at 37° C.

### 6.9.13 Drug Release Profile

The drug release profile of EBNPs under different pH value (7.4 and 5.0) at 37° C. was determined by dialysis method. 500 μL of EBNPs were dialyzed in a 3500 Da-cutoff dialysis bag against 4 mL of PBS (pH 7.4 and pH 5.0). The outer solution was completely replaced at each time and fresh PBS was subsequently added. The cumulative release percentage of EIPA and BTZ over the time was calculated by HPLC measurement.

### 6.9.14 Animal Studies

Female BALB/c nude mice (4-6 weeks) were purchased from Centre for Comparative Medicine Research (Li Ka Shing Faculty of Medicine, The University of Hong Kong). All animal received care and experiments based on the protocol that was approved by the Committee on the Use of Live Animals in Teaching and Research (CULATR) at Li Ka Shing Faculty of Medicine (CULATR No. 22-200). Animals were maintained at the conventional experimental holding area Dexter H. C. Man Building at the Centre for Comparative Medicine Research. To construct A549 xenograft breast cancer model, 300×10⁶ of A549 cells in DMEM, supplemented with Matrigel (Corning, 354248) and collagen I (Gibco, A1048301) were subcutaneously implanted in the right flank of the mice. The treatment procedure was initiated when tumor volumes reached about 100 mm³.

### 6.9.15 Anti-Tumor Efficacy Study

The tumor-bearing mice were divided into seven groups (n=4 per group). These seven groups of mice were treated with EIPA, BTZ, EIPA+BTZ, ENP, BNP, and EBNP. The concentration of EIPA and BTZ in different formulations were 3 mg/kg and 0.75 mg/kg, respectively. Different formulations were intravenously injected into the mice six times at an interval of three days. The body weight and tumor size of mice were measured every 2 days. Finally, after a 22-day treatment, the mice were euthanized to isolate the tumor tissues for further analysis.

### 6.9.16 H&E Staining

The tumor tissues and major organs (heart, liver, spleen, lung, and kidney) of these mice were harvested and H&E staining was performed to evaluate the anti-cancer effect and biosafety profile, respectively. Briefly, 4 mm paraffin sections were dried under 60° C. for 2 h, dewaxed in xylene and rehydrated in gradient concentrations of ethanol step by step. The slides were stained with Mayers Hematoxylin for 1 min, washed in running tap water, acid ethanol and deionized water, sequentially. Then the slides were stained with Alcoholic-Eosin for 1 min. Subsequently, the slides were dehydrated and rinsed in several baths of xylene and a thin layer of polystyrene mountant was applied, followed by a glass coverslip.

### 6.9.17 Bioinformatic Analysis

The RNAseq data from GDC TCGA lung adenocarcinoma (LUAD) patient cohort were collected for analysis (tcga-data.nci.nih.gov/). The data were processed under R environment (version 4.1.2, https://www.r-project.org/). The samples were divided into two groups, tumor tissues or adjacent normal tissues. We collected the UPS-related gene set containing 676 UPS-correlated genes according to a previous study and conducted differential expression genes (DEGs) analysis based on this data set, using the DESeq2 package in R (available at https://bioconductor.org/packages/3.16/bioc/html/DESeq2.html) (2, 3). Gene ontology (GO) enrichment analysis was further conducted to define the correlation of these DEGs with UPS system. The “GOplot” package was employed to visualize the enrichment results (4). Univariate Cox regression analysis was performed to identify prognosis-related UPS genes. And among these genes, Lasso regression analysis was further utilized to define independent prognostic genes.

Hence, a prognostic model was constructed, and patients were divided into two groups of low risk or high risk according to the expression of these genes. Then survival analysis was performed by Kaplan-Meier method. For the RNAseq analysis in vitro, normal A549 and starvation-adapted A549 cells were collected and total RNA was extracted using Qiagen RNeasy Mini Kit (Qiagen, 74104). DEGs analysis was performed and heatmap plot was generated using pheatmap package. GSEA analysis was performed to validate the correlation of DEGs and UPS activity in GSEA software (version 4.0.1).

### 6.9.18 Statistical Analysis

GraphPad Prism 8.0 software (GraphPad Software, Inc) was used for statistical data analysis. To compare the differences between two groups, two-tailed unpaired Student's t-test was used. To analyze and compare the differences among multiple-group means, one-way analysis of variance (ANOVA) with Tukey's multiple comparison test was applied. For anti-cancer effect in vivo, two-way ANOVA with Tukey's multiple comparison analysis was employed. Values of P<0.05 were considered significant. Results are represented as means±SD. *p<0.05, **p<0.01, ***p<0.001, ****p<0.0001.

### Exemplary Products, Systems and Methods are Set Out in the Following Items:

1. A method of treating cancer, comprising the step of administering to a subject in need thereof a combination of: (i) a ubiquitin-proteasome system (UPS) inhibitor; and (ii) a macropinocytosis inhibitor.

2. The method of item 1, wherein the ubiquitin-proteasome system (UPS) inhibitor is selected from the group consisting of bortezomib (BTZ), carfilzomib, ixazomib, lactacystin, disulfiram, epigallocatechin-3-gallate, marizomib (salinosporamide A), oprozomib (ONX-0912), delanzomib (CEP-18770), epoxomicin, MG132, and beta-hydroxy beta-methylbutyrate.

3. The method of any preceding item, wherein the ubiquitin-proteasome system (UPS) inhibitor is bortezomib.

4. The method of any preceding item, wherein the macropinocytosis inhibitor is selected from the group consisting of 5-(n-ethyl-n-isopropyl)-amiloride (EIPA), amiloride, imipramine, phenoxybenzamine, vinblastine, wortmannin, latrunculin A (Lat A), and latrunculin B (Lat B).

5. The method of any preceding item, wherein the subject has a tumor, wherein the tumor is present in or surrounded by a nutrient-deprived microenvironment.

6. The method of any preceding item, wherein the tumor microenvironment is deprived of amino acids.

7. The method of any preceding item, wherein the amino-acid deprived tumor microenvironment facilitates protein internalization into a tumor cell.

8. The method of any preceding item, wherein the macropinocytosis inhibitor inhibits said protein internalization.

9. The method of any preceding item, wherein the amino acid-deprived tumor microenvironment activates ubiquitin-proteasome system (UPS)-dependent protein degradation in a tumor cell.

10. The method of any preceding item, wherein the ubiquitin-proteasome system (UPS) inhibitor inhibits said UPS-dependent protein degradation.

11. The method of any preceding item, wherein the combination of the ubiquitin-proteasome system (UPS) inhibitor and the macropinocytosis inhibitor inhibits protein internalization and UPS-dependent protein degradation in said tumor.

12. The method of any preceding item, wherein the ubiquitin-proteasome system (UPS) inhibitor and the macropinocytosis inhibitor have a synergistic effect.

13. The method of any preceding item, wherein the ubiquitin-proteasome system (UPS) inhibitor and the macropinocytosis inhibitor have an additive effect.

14. The method of any preceding item, wherein the combination increases apoptosis of tumor cells.

15. The method of any preceding item, wherein the ubiquitin-proteasome system (UPS) inhibitor and macropinocytosis inhibitor are provided in a pharmaceutical composition.

16. The method of any preceding item, wherein the ubiquitin-proteasome system (UPS) inhibitor and macropinocytosis inhibitor are co-encapsulated in a polymersome nanoparticle.

17. The method of any preceding item, wherein the polymersome nanoparticle comprises a hydrophilic core and a hydrophobic membrane bilayer.

18. The method of any preceding item, wherein the polymersome nanoparticle comprises a pH-responsive moiety.

19. The method of any preceding item, wherein the pH-sensitive moiety comprises an acrylate, a methacrylate, an acetate or a phthalate moiety.

20. The method of any preceding item, wherein the pH-sensitive moiety is selected from the group consisting of tetrahydropyranyl methacrylate (THPMA), tetrahydropyranyl-2-methyl methacrylate (THPMM), 2-(diethylamino)-ethyl acrylate, N,N-dimethylaminoethyl methacrylate (DMEEMA), 2-(tert-butylamino)-ethyl methacrylate (tBuMAEMA), N,N-diethylaminoethyl methacrylate (DEAEMA), and 2-(diisopropylamino)-ethyl methacrylate (DIPAEMA), hydroxypropyl-methylcellulose phthalate, and HPMC acetate succinate (HPMC-AS).

21. The method of any preceding item, wherein the pH-sensitive moiety is tetrahydropyranyl methacrylate (THPMA).

22. The method of any preceding item, wherein the polymersome comprises a benzyl methacrylate (BzMA) moiety.

23. The method of any preceding item, wherein the polymersome nanoparticle comprises one or more polymers selected from the group consisting of polytetrahydropyranyl methacrylate (PTHPMA), polybenzyl methacrylate (PBzMA), polyacrylic acid, polymethacrylic acid, polyaminoalkyl acrylate, polyaminoalkyl methacrylate, polytetrahydropyranyl-2-methyl methacrylate (PTHPMM), poly-(2-(diethylamino)ethyl) methacrylate, poly-N,N-dimethylaminoethyl methacrylate (PDMEEMA), poly-(2-(tert-butylamino)-ethyl) methacrylate (PtBuMAEMA), poly-N,N-diethylaminoethyl methacrylate (PDEAEMA), poly-(2-(diisopropylamino)ethyl) methacrylate (PDIPAEMA) and/or copolymers thereof.

24. The method of any preceding item, wherein the polymersome nanoparticle comprises a polyethylene glycol (PEG) or polypropylene glycol (PPG) moiety.

25. The method of any preceding item, wherein the polymersome nanoparticle comprises a block copolymer.

26. The method of any preceding item, wherein the block copolymer is selected from the group consisting of polytetrahydropyranyl methacrylate-polybenzyl methacrylate (P(BzMA-co-THPMA), polyethylene glycol (PEG)-b-P(BzMA-co-THPMA), poly(methyl methacrylate)-b-poly(acrylic acid) (PMMA-b-PAA), poly(methyl methacrylate)-poly(t-butyl acrylate) (PMMA-b-PtBA), poly(methacrylic acid, methyl methacrylate, poly(methacrylic acid, methyl methacrylate), poly(methacrylic acid, ethyl acrylate), poly(butyl methacrylate, (2-dimethylaminoethyl) methacrylate, methyl methacrylate), poly(methyl acrylate, methyl methacrylate, methacrylic acid), poly(ethyl acrylate, methyl methacrylate, trimethylammonioethyl methacrylate chloride), polyethylene glycol-b-polycaprolacton (PEG-b-PCL), polyethylene glycol-b-polylactide (PEG-b-PLA), polyethylene glycol-b-poly(lactic-co-glycolic acid) (PEG-b PLGA), polyethylene glycol-b-polyglycolid (PEG-b PGA), poly(dimethylsiloxane)-b-poly(2-methyloxazoline) (PDMS-b-PMOXA), poly(3-caprolactone) b-poly(2-methacryloyloxyethylphosphorylcholine) (PCL-b-PMPC), polylactid-b-poly(2-methacryloyloxyethylphosphorylcholine) (PLA-b-PMPC), polyethylene glycol-b-polybutadiene (PEG b-PBD), polyethylene glycol-b-polyethylethylene (PEG-b-PEE), polyethylene glycol-b-polyphenylene sulfide (PEG-b-PPS), polyethylene glycol-b-polytrimethylene carbonate (PEG-b-PTMC), poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG).

27. A polymersome nanoparticle comprising a ubiquitin-proteasome system (UPS) inhibitor and a macropinocytosis inhibitor.

28. The nanoparticle of any preceding item, wherein the ubiquitin-proteasome system (UPS) inhibitor and macropinocytosis inhibitor are co-encapsulated in the polymersome nanoparticle.

29. The nanoparticle of any preceding item, wherein the polymersome nanoparticle comprises a hydrophilic core and a hydrophobic membrane bilayer.

30. The nanoparticle of any preceding item, wherein the polymersome nanoparticle comprises a pH-responsive moiety.

31. The nanoparticle of any preceding item, wherein the pH-sensitive moiety comprises an acrylate, a methacrylate, an acetate or a phthalate moiety.

32. The nanoparticle of any preceding item, wherein the pH-sensitive moiety is selected from the group consisting of tetrahydropyranyl methacrylate (THPMA), tetrahydropyranyl-2-methyl methacrylate (THPMM), 2-(diethylamino)ethyl acrylate, N,N-dimethylaminocthyl methacrylate (DMEEMA), 2-(tert-butylamino)-ethyl methacrylate (tBuMAEMA), N,N-diethylaminoethyl methacrylate (DEAEMA), and 2-(diisopropylamino)ethyl methacrylate (DIPAEMA), hydroxypropyl-methylcellulose phthalate, and HPMC acetate succinate (HPMC-AS).

33. The nanoparticle of any preceding item, wherein the pH-sensitive moiety is tetrahydropyranyl methacrylate (THPMA).

34. The nanoparticle of any preceding item, wherein the polymersome comprises a benzyl methacrylate (BzMA) moiety.

35. The nanoparticle of any preceding item, wherein the polymersome nanoparticle comprises one or more polymers selected from the group consisting of polytetrahydropyranyl methacrylate (PTHPMA), polybenzyl methacrylate (PBzMA), polyacrylic acid, polymethacrylic acid, polyaminoalkyl acrylate, polyaminoalkyl methacrylate, polytetrahydropyranyl-2-methyl methacrylate (PTHPMM), poly-(2-(diethylamino)ethyl) methacrylate, poly-N,N-dimethylaminoethyl methacrylate (PDMEEMA), poly-(2-(tert-butylamino)-ethyl) methacrylate (PtBuMAEMA), poly-N,N-diethylaminoethyl methacrylate (PDEAEMA), poly-(2-(diisopropylamino)ethyl) methacrylate (PDIPAEMA) and/or copolymers thereof.

36. The nanoparticle of any preceding item, wherein the polymersome nanoparticle comprises a polyethylene glycol (PEG) or polypropylene glycol (PPG) moiety.

37. The nanoparticle of any preceding item, wherein the polymersome nanoparticle comprises a block copolymer.

38. The nanoparticle of any preceding item, wherein the block copolymer is selected from the group consisting of polytetrahydropyranyl methacrylate-polybenzyl methacrylate (P(BzMA-co-THPMA), polyethylene glycol (PEG)-b-P(BzMA-co-THPMA), poly(methyl methacrylate)-b-poly(acrylic acid) (PMMA-b-PAA), poly(methyl methacrylate)-poly(t-butyl acrylate) (PMMA-b-PtBA), poly(methacrylic acid, methyl methacrylate, poly(methacrylic acid, methyl methacrylate), poly(methacrylic acid, ethyl acrylate), poly(butyl methacrylate, (2-dimethylaminoethyl) methacrylate, methyl methacrylate), poly(methyl acrylate, methyl methacrylate, methacrylic acid), poly(ethyl acrylate, methyl methacrylate, trimethylammonioethyl methacrylate chloride), polyethylene glycol-b-polycaprolacton (PEG-b-PCL), polyethylene glycol-b-polylactide (PEG-b-PLA), polyethylene glycol-b-poly(lactic-co-glycolic acid) (PEG-b PLGA), polyethylene glycol-b-polyglycolid (PEG-b PGA), poly(dimethylsiloxane)-b-poly(2-methyloxazoline) (PDMS-b-PMOXA), poly(3-caprolactone) b-poly(2-methacryloyloxyethylphosphorylcholine) (PCL-b-PMPC), polylactid-b-poly(2-methacryloyloxyethylphosphorylcholine) (PLA-b-PMPC), glycol-b-polyethylene glycol-b-polybutadiene (PEG b-PBD), polyethylene polyethylethylene (PEG-b-PEE), polyethylene glycol-b-polyphenylene sulfide (PEG-b-PPS), polyethylene glycol-b-polytrimethylene carbonate (PEG-b-PTMC), poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG).

39. A method of treating a tumor, comprising the step of contacting the tumor with a combination of (i) a ubiquitin-proteasome system (UPS) inhibitor; and (ii) a macropinocytosis inhibitor.

40. A method of reducing protein internalization and degradation in a tumor surrounded by an amino-acid deprived microenvironment, the method comprises the step of contacting the tumor with a combination of (i) a ubiquitin-proteasome system (UPS) inhibitor; and (ii) a macropinocytosis inhibitor.

41. A method of reducing protein internalization and degradation in a tumor surrounded by an amino-acid deprived microenvironment, the method comprises the step of contacting the tumor with a polymersome nanoparticle comprising a combination of (i) a ubiquitin-proteasome system (UPS) inhibitor; and (ii) a macropinocytosis inhibitor.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

### REFERENCES

1. M. Pan, C. Zorbas, M. Sugaya, K. Ishiguro, M. Kato, M. Nishida, H.-F. Zhang, M. M. Candeias, A. Okamoto, T. Ishikawa, T. Soga, H. Aburatani, J. Sakai, Y. Matsumura, T. Suzuki, C. G. Proud, D. L. J. Lafontaine, T. Osawa, Glutamine deficiency in solid tumor cells confers resistance to ribosomal RNA synthesis inhibitors. Nature Communications 13, 3706 (2022).
2. W. Palm, Y. Park, K. Wright, N. N. Pavlova, D. A. Tuveson, C. B. Thompson, The Utilization of Extracellular Proteins as Nutrients Is Suppressed by mTORC1. Cell 162, 259-270 (2015).
3. G. Leprivier, M. Remke, B. Rotblat, A. Dubuc, A.-Rachele F. Mateo, M. Kool, S. Agnihotri, A. El-Naggar, B. Yu, S. Prakash Somasekharan, B. Faubert, G. Bridon, Cristina E. Tognon, J. Mathers, R. Thomas, A. Li, A. Barokas, B. Kwok, M. Bowden, S. Smith, X. Wu, A. Korshunov, T. Hielscher, Paul A. Northcott, Jason D. Galpin, Christopher A. Ahern, Y. Wang, Martin G. McCabe, V. P. Collins, Russell G. Jones, M. Pollak, O. Delattre, Martin E. Gleave, E. Jan, Stefan M. Pfister, Christopher G. Proud, W. B. Derry, Michael D. Taylor, Poul H. Sorensen, The eEF2 Kinase Confers Resistance to Nutrient Deprivation by Blocking Translation Elongation. Cell 153, 1064-1079 (2013).
4. C. Commisso, S. M. Davidson, R. G. Soydaner-Azeloglu, S. J. Parker, J. J. Kamphorst, S. Hackett, E. Grabocka, M. Nofal, J. A. Drebin, C. B. Thompson, J. D. Rabinowitz, C. M. Metallo, M. G. Vander Heiden, D. Bar-Sagi, Macropinocytosis of protein is an amino acid supply route in Ras-transformed cells. Nature 497, 633-637 (2013).
5. E. Ratto, S. R. Chowdhury, N. S. Siefert, M. Schneider, M. Wittmann, D. Helm, W. Palm, Direct control of lysosomal catabolic activity by mTORC1 through regulation of V-ATPase assembly. Nature Communications 13, 4848 (2022).
6. N. Mizushima, The role of the Atg1/ULK1 complex in autophagy regulation. Current Opinion in Cell Biology 22, 132-139 (2010).
7. Z. Sha, J. Zhao, A. L. Goldberg, Measuring the Overall Rate of Protein Breakdown in Cells and the Contributions of the Ubiquitin-Proteasome and Autophagy-Lysosomal Pathways. Methods Mol Biol 1844, 261-276 (2018).
8. K. L. Rock, C. Gramm, L. Rothstein, K. Clark, R. Stein, L. Dick, D. Hwang, A. L. Goldberg, Inhibitors of the proteasome block the degradation of most cell proteins and the generation of peptides presented on MHC class I molecules. Cell 78, 761-771 (1994).
9. B. Gong, M. Radulovic, M. E. Figueiredo-Pereira, C. Cardozo, The Ubiquitin-Proteasome System: Potential Therapeutic Targets for Alzheimer's Disease and Spinal Cord Injury. Front Mol Neurosci 9, 4 (2016).
10. A. Varshavsky, Regulated protein degradation. Trends Biochem Sci 30, 283-286 (2005).
11. H. C. Tai, E. M. Schuman, Ubiquitin, the proteasome and protein degradation in neuronal function and dysfunction. Nat Rev Neurosci 9, 826-838 (2008).
12. M. Nofal, K. Zhang, S. Han, J. D. Rabinowitz, mTOR Inhibition Restores Amino Acid Balance in Cells Dependent on Catabolismof Extracellular Protein. Mol Cell 67, 936-946 e935 (2017).
13. S. Lokireddy, N. V. Kukushkin, A. L. Goldberg, cAMP-induced phosphorylation of 26S proteasomes on Rpn6/PSMD11 enhances their activity and the degradation of misfolded proteins. Proceedings of the National Academy of Sciences 112, E7176-E7185 (2015).
14. F. A. Mallette, S. Richard, K48-linked ubiquitination and protein degradation regulate 53BP1 recruitment at DNA damage sites. Cell Research 22, 1221-1223 (2012).
15. W. Tinganelli, M. Durante, Tumor Hypoxia and Circulating Tumor Cells. Int J Mol Sci 21, (2020).
16. X. M. Ma, J. Blenis, Molecular mechanisms of mTOR-mediated translational control. Nature Reviews Molecular Cell Biology 10, 307-318 (2009).
17. Y. Sancak, T. R. Peterson, Y. D. Shaul, R. A. Lindquist, C. C. Thoreen, L. Bar-Peled, D. M. Sabatini, The Rag GTPases Bind Raptor and Mediate Amino Acid Signaling to mTORC1. Science 320, 1496-1501 (2008).
18. J. Zhao, B. Zhai, S. P. Gygi, A. L. Goldberg, mTOR inhibition activates overall protein degradation by the ubiquitin proteasome system as well as by autophagy. Proceedings of the National Academy of Sciences 112, 15790-15797 (2015).
19. N. Wang, QilianPeng, PanpanYu, YunhaoXu, ShiqiangWang, GuanghuiYing, Zheng Wang, Hongfeng, Autophagy and Ubiquitin-Proteasome System Coordinate to Regulate the Protein Quality Control of Neurodegenerative Disease-Associated DCTN1. Neurotoxicity research 37, (2020).
20. Z. Huang, Y. Wu, X. Zhou, J. Xu, W. Zhu, Y. Shu, P. Liu, Efficacy of therapy with bortezomib in solid tumors: a review based on 32 clinical trials. Future Oncol 10, 1795-1807 (2014).
21. S. Zheng, W. Wang, J. Aldahdooh, A. Malyutina, T. Shadbahr, Z. Tanoli, A. Pessia, J. Tang, SynergyFinder Plus: Toward Better Interpretation and Annotation of Drug Combination Screening Datasets. Genomics Proteomics Bioinformatics, (2022).
22. J. Wu, The Enhanced Permeability and Retention (EPR) Effect: The Significance of the Concept and Methods to Enhance Its Application. J Pers Med 11, (2021).
23. F. Xu, X. Li, X. Huang, J. Pan, Y. Wang, S. Zhou, Development of a pH-responsive polymersome inducing endoplasmic reticulum stress and autophagy blockade. Science Advances 6, cabb8725.
24. J. Jung, I.-H. Lee, E. Lee, J. Park, S. Jon, pH-Sensitive Polymer Nanospheres for Use as a Potential Drug Delivery Vehicle. Biomacromolecules 8, 3401-3407 (2007).
25. R. G. Jones, C. B. Thompson, Tumor suppressors and cell metabolism: a recipe for cancer growth. Genes Dev 23, 537-548 (2009).
26. C. Pechincha, S. Groessl, R. Kalis, M. de Almeida, A. Zanotti, M. Wittmann, M. Schneider, R. P. de Campos, S. Rieser, M. Brandstetter, A. Schleiffer, K. Muller-Decker, D. Helm, S. Jabs, D. Haselbach, M. K. Lemberg, J. Zuber, W. Palm, Lysosomal enzyme trafficking factor LYSET enables nutritional usage of extracellular proteins. Science 378, eabn5637 (2022).
27. V. Cohen-Kaplan, I. Livneh, N. Avni, B. Fabre, T. Ziv, Y. T. Kwon, A. Ciechanover, p62- and ubiquitin-dependent stress-induced autophagy of the mammalian 26S proteasome. Proceedings of the National Academy of Sciences 113, E7490-E7499 (2016).
28. V. Jayashankar, A. L. Edinger, Macropinocytosis confers resistance to therapies targeting cancer anabolism. Nat Commun 11, 1121 (2020).
29. S. Kageyama, Y.-s. Sou, T. Uemura, S. Kametaka, T. Saito, R. Ishimura, T. Kouno, L. Bedford, R. J. Mayer, M.-S. Lee, M. Yamamoto, S. Waguri, K. Tanaka, M. Komatsu, Proteasome Dysfunction Activates Autophagy and the Keap1-Nrf2 Pathway *. Journal of Biological Chemistry 289, 24944-24955 (2014).
30. R. D. Al Bostami, W. H. Abuwatfa, G. A. Husseini, Recent Advances in Nanoparticle-Based Co-Delivery Systems for Cancer Therapy. Nanomaterials (Basel) 12, (2022).
31. Y. Nakamura, A. Mochida, P. L. Choyke, H. Kobayashi, Nanodrug Delivery: Is the Enhanced Permeability and Retention Effect Sufficient for Curing Cancer? Bioconjug Chem 27, 2225-2238 (2016).
32. D. García Jiménez, M. Rossi Sebastiano, M. Vallaro, V. Mileo, D. Pizzirani, E. Moretti, G. Ermondi, G. Caron, Designing Soluble PROTACs: Strategies and Preliminary Guidelines. Journal of Medicinal Chemistry 65, 12639-12649 (2022).
33. J. Lee, Y. M. Lee, J. Kim, W. J. Kim, Doxorubicin/Ce6-Loaded Nanoparticle Coated with Polymer via Singlet Oxygen-Sensitive Linker for Photodynamically Assisted Chemotherapy. Nanotheranostics 1, 196-207 (2017).
34. Y. Ma, Y. Zhao, N. K. Bejjanki, X. Tang, W. Jiang, J. Dou, M. I. Khan, Q. Wang, J. Xia, H. Liu, Y.-Z. You, G. Zhang, Y. Wang, J. Wang, Nanoclustered Cascaded Enzymes for Targeted Tumor Starvation and Deoxygenation-Activated Chemotherapy without Systemic Toxicity. ACS Nano 13, 8890-8902 (2019).
35. L. Kou, X. Jiang, Y. Tang, X. Xia, Y. Li, A. Cai, H. Zheng, H. Zhang, V. Ganapathy, Q. Yao, R. Chen, Resetting amino acid metabolism of cancer cells by ATB (0,+)-targeted nanoparticles for enhanced anticancer therapy. Bioact Mater 9, 15-28 (2022).
36. T. Wang, K. Long, Y. Zhou, X. Jiang, J. Liu, J. H. C. Fong, A. S. L. Wong, W.-L. Ng, W. Wang, Optochemical Control of mTOR Signaling and mTOR-Dependent Autophagy. ACS Pharmacology & Translational Science 5, 149-155 (2022).
37. M. I. Love, W. Huber, S. Anders, Moderated estimation of fold change and dispersion for RNA-seq data with DESeq2. Genome Biology 15, 550 (2014).
38. H. Guo, Y. Li, Y. Liu, L. Chen, Z. Gao, L. Zhang, N. Zhou, H. Guo, B. Shi, Prognostic Role of the Ubiquitin Proteasome System in Clear Cell Renal Cell Carcinoma: A Bioinformatic Perspective. J Cancer 12, 4134-4147 (2021).
39. W. Walter, F. Sánchez-Cabo, M. Ricote, GOplot: an R package for visually combining expression data with functional analysis. Bioinformatics 31, 2912-2914 (2015).

What is claimed:

1. A method of treating cancer, comprising the step of administering to a subject in need thereof a composition having a polymersome nanoparticle, the nanoparticle comprising a combination of: (i) a ubiquitin-proteasome system (UPS) inhibitor; and (ii) a macropinocytosis inhibitor.

2. The method of claim 1, wherein the ubiquitin-proteasome system (UPS) inhibitor is selected from the group consisting of bortezomib (BTZ), carfilzomib, ixazomib, lactacystin, disulfiram, epigallocatechin-3-gallate, marizomib (salinosporamide A), oprozomib (ONX-0912), delanzomib (CEP-18770), epoxomicin, MG132, and beta-hydroxy beta-methylbutyrate.

3. The method of claim 1, wherein the ubiquitin-proteasome system (UPS) inhibitor is bortezomib.

4. The method of claim 1, wherein the macropinocytosis inhibitor is selected from the group consisting of 5-(n-ethyl-n-isopropyl)-amiloride (EIPA), amiloride, imipramine, phenoxybenzamine, vinblastine, wortmannin, latrunculin A (Lat A), and latrunculin B (Lat B).

5. The method of claim 1, wherein the subject has a tumor, wherein the tumor is present in or surrounded by a nutrient-deprived microenvironment.

6. The method of claim 5, wherein the tumor microenvironment is deprived of amino acids.

7. The method of claim 6, wherein the amino-acid deprived tumor microenvironment facilitates protein internalization into a tumor cell.

8. The method of claim 7, wherein the macropinocytosis inhibitor inhibits said protein internalization.

9. The method of claim 1, wherein the combination of the ubiquitin-proteasome system (UPS) inhibitor and the macropinocytosis inhibitor inhibits protein internalization and UPS-dependent protein degradation in said tumor.

10. The method of claim 1, wherein the ubiquitin-proteasome system (UPS) inhibitor and the macropinocytosis inhibitor have a synergistic effect or an additive effect.

11. The method of claim 1, wherein the combination increases apoptosis of tumor cells.

12. The method of claim 1, wherein the ubiquitin-proteasome system (UPS) inhibitor and macropinocytosis inhibitor are co-encapsulated respectively in a hydrophilic core and a hydrophobic membrane bilayer of a polymersome nanoparticle.

13. The method of claim 12, wherein the polymersome nanoparticle comprises a pH-responsive moiety.

14. The method of claim 13, wherein the pH-sensitive moiety comprises an acrylate, a methacrylate, an acetate or a phthalate moiety.

15. The method of claim 13, wherein the pH-sensitive moiety is selected from the group consisting of tetrahydropyranyl methacrylate (THPMA), tetrahydropyranyl-2-methyl methacrylate (THPMM), 2-(diethylamino)ethyl acrylate, N,N-dimethylaminoethyl methacrylate (DMEEMA), 2-(tert-butylamino)-ethyl methacrylate (tBuMAEMA), N,N-diethylaminoethyl methacrylate (DEAEMA), and 2-(diisopropylamino)ethyl methacrylate (DIPAEMA), hydroxypropyl-methylcellulose phthalate, and HPMC acetate succinate (HPMC-AS).

16. The method of claim 13, wherein the pH-sensitive moiety is tetrahydropyranyl methacrylate (THPMA).

17. The method of claim 13, wherein the polymersome comprises a benzyl methacrylate (BzMA) moiety.

18. The method of claim 1, wherein the polymersome nanoparticle comprises one or more polymers selected from the group consisting of polytetrahydropyranyl methacrylate (PTHPMA), polybenzyl methacrylate (PBzMA), polyacrylic acid, polymethacrylic acid, polyaminoalkyl acrylate, polyaminoalkyl methacrylate, polytetrahydropyranyl-2-methyl methacrylate (PTHPMM), poly-(2-(diethylamino)ethyl) methacrylate, poly-N,N-dimethylaminoethyl methacrylate (PDMEEMA), poly-(2-(tert-butylamino)-ethyl) methacrylate (PtBuMAEMA), poly-N,N-diethylaminoethyl methacrylate (PDEAEMA), poly-(2-(diisopropylamino)ethyl) methacrylate (PDIPAEMA) and/or copolymers thereof.

19. The method of claim 1, wherein the polymersome nanoparticle comprises a polyethylene glycol (PEG) or polypropylene glycol (PPG) moiety.

20. The method of claim 1, wherein the polymersome nanoparticle comprises a block copolymer.

21. The method of claim 20, wherein the block copolymer is selected from the group consisting of polytetrahydropyranyl methacrylate-polybenzyl methacrylate (P(BzMA-co-THPMA), polyethylene glycol (PEG)-b-P(BzMA-co-THPMA), poly(methyl methacrylate)-b-poly(acrylic acid) (PMMA-b-PAA), poly(methyl methacrylate)-poly(t-butyl acrylate) (PMMA-b-PtBA), poly(methacrylic acid, methyl methacrylate, poly(methacrylic acid, methyl methacrylate), poly(methacrylic acid, ethyl acrylate), poly(butyl methacrylate, (2-dimethylaminoethyl) methacrylate, methyl methacrylate), poly(methyl acrylate, methyl methacrylate, methacrylic acid), poly(ethyl acrylate, methyl methacrylate, trimethylammonioethyl methacrylate chloride), polyethylene glycol-b-polycaprolacton (PEG-b-PCL), polyethylene glycol-b-polylactide (PEG-b-PLA), polyethylene glycol-b-poly(lactic-co-glycolic acid) (PEG-b PLGA), polyethylene glycol-b-polyglycolid (PEG-b PGA), poly(dimethylsiloxane)-b-poly(2-methyloxazoline) (PDMS-b-PMOXA), poly(3-caprolactone) b-poly(2-methacryloyloxyethylphosphorylcholine) (PCL-b-PMPC), polylactid-b-poly(2-methacryloyloxyethylphosphorylcholine) (PLA-b-PMPC), polyethylene glycol-b-polybutadiene (PEG b-PBD), polyethylene glycol-b-polyethylethylene (PEG-b-PEE), polyethylene glycol-b-polyphenylene sulfide (PEG-b-PPS), polyethylene glycol-b-polytrimethylene carbonate (PEG-b-PTMC), poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG).

22. A polymersome nanoparticle comprising a ubiquitin-proteasome system (UPS) inhibitor and a macropinocytosis inhibitor.

23. The nanoparticle of claim 22, wherein the ubiquitin-proteasome system (UPS) inhibitor and macropinocytosis inhibitor are co-encapsulated respectively in a hydrophilic core and a hydrophobic bilayer of the polymersome nanoparticle.

24. The nanoparticle of claim 22, wherein the polymersome nanoparticle comprises a pH-responsive moiety.

25. The nanoparticle of claim 24, wherein the pH-sensitive moiety comprises an acrylate, a methacrylate, an acetate or a phthalate moiety.

26. The nanoparticle of claim 24, wherein the pH-sensitive moiety is selected from the group consisting of tetrahydropyranyl methacrylate (THPMA), tetrahydropyranyl-2-methyl methacrylate (THPMM), 2-(diethylamino)ethyl acrylate, N,N-dimethylaminocthyl methacrylate (DMEEMA), 2-(tert-butylamino)-ethyl methacrylate (tBuMAEMA), N,N-diethylaminoethyl methacrylate (DEAEMA), and 2-(diisopropylamino)ethyl methacrylate (DIPAEMA), hydroxypropyl-methylcellulose phthalate, and HPMC acetate succinate (HPMC-AS).

27. The nanoparticle of claim 24, wherein the pH-sensitive moiety is tetrahydropyranyl methacrylate (THPMA).

28. The nanoparticle of claim 22, wherein the polymersome comprises a benzyl methacrylate (BzMA) moiety.

29. The nanoparticle of claim 22, wherein the polymersome nanoparticle comprises one or more polymers selected from the group consisting of polytetrahydropyranyl methacrylate (PTHPMA), polybenzyl methacrylate (PBzMA), polyacrylic acid, polymethacrylic acid, polyaminoalkyl acrylate, polyaminoalkyl methacrylate, polytetrahydropyranyl-2-methyl methacrylate (PTHPMM), poly-(2-(diethylamino)ethyl) methacrylate, poly-N,N-dimethylaminoethyl methacrylate (PDMEEMA), poly-(2-(tert-butylamino)-ethyl) methacrylate (PtBuMAEMA), poly-N,N-diethylaminoethyl methacrylate (PDEAEMA), poly-(2-(diisopropylamino)ethyl) methacrylate (PDIPAEMA) and/or copolymers thereof.

30. The nanoparticle of claim 22, wherein the polymersome nanoparticle comprises a polyethylene glycol (PEG) or polypropylene glycol (PPG) moiety.

31. The nanoparticle of claim 22, wherein the polymersome nanoparticle comprises a block copolymer.

32. The nanoparticle of claim 22, wherein the block copolymer is selected from the group consisting of polytetrahydropyranyl methacrylate-polybenzyl methacrylate (P(BzMA-co-THPMA), polyethylene glycol (PEG)-b-P(BzMA-co-THPMA), poly(methyl methacrylate)-b-poly(acrylic acid) (PMMA-b-PAA), poly(methyl methacrylate)-poly(t-butyl acrylate) (PMMA-b-PtBA), poly(methacrylic acid, methyl methacrylate, poly(methacrylic acid, methyl methacrylate), poly(methacrylic acid, ethyl acrylate), poly(butyl methacrylate, (2-dimethylaminoethyl) methacrylate, methyl methacrylate), poly(methyl acrylate, methyl methacrylate, methacrylic acid), poly(ethyl acrylate, methyl methacrylate, trimethylammonioethyl methacrylate chloride), polyethylene glycol-b-polycaprolacton (PEG-b-PCL), polyethylene glycol-b-polylactide (PEG-b-PLA), polyethylene glycol-b-poly(lactic-co-glycolic acid) (PEG-b PLGA), polyethylene glycol-b-polyglycolid (PEG-b PGA), poly(dimethylsiloxane)-b-poly(2-methyloxazoline) (PDMS-b-PMOXA), poly(3-caprolactone) b-poly(2-methacryloyloxyethylphosphorylcholine) (PCL-b-PMPC), polylactid-b-poly(2-methacryloyloxyethylphosphorylcholine) (PLA-b-PMPC), polyethylene glycol-b-polybutadiene (PEG b-PBD), polyethylene glycol-b-polyethylethylene (PEG-b-PEE), polyethylene glycol-b-polyphenylene sulfide (PEG-b-PPS), polyethylene glycol-b-polytrimethylene carbonate (PEG-b-PTMC), poly(lactic-co-glycolic acid)-b-polyethylene glycol-poly(lactic-co-glycolic acid) (PLGA-PEG-PLGA), poly(dimethylsiloxane)-b-poly(2-methyloxazoline)-b-poly(dimethylsiloxane) (PMOXA-b-PDMS-b-PMOXA), polyethylene glycol-b-polypropylene glycol-b-polyethylene glycol (PEG-PPO-PEG).

33. A method of reducing protein internalization and degradation in a tumor surrounded by an amino-acid deprived microenvironment, the method comprises the step of contacting the tumor with a polymersome nanoparticle comprising a combination of (i) a ubiquitin-proteasome system (UPS) inhibitor; and (ii) a macropinocytosis inhibitor.
